(19) United States
(12) Patent Application Publication
(10) Pub. No.: US 2014/0227757 A1
(43) Pub. Date: Aug. 14, 2014

(54) INTEGRATED PROCESSES FOR CONVERSION OF LIGNOCELLULOSIC BIOMASS TO BIOPRODUCTS AND SYSTEMS AND APPARATUS RELATED THERETO

(71) Applicant: BOARD OF TRUSTEES OF MICHIGAN STATE UNIVERSITY, East Lansing, MI (US)

(72) Inventors: Mingjie Jin, Lansing, MI (US); Venkatesh Balan, East Lansing, MI (US); Bruce E. Dale, Mason, MI (US)

(73) Assignee: BOARD OF TRUSTEES OF MICHIGAN STATE UNIVERSITY, East Lansing, MI (US)

(21) Appl. No.: 14/251,921

(22) Filed: Apr. 14, 2014

Related U.S. Application Data
(63) Continuation of application No. PCT/US2012/059898, filed on Oct. 12, 2012.
(60) Provisional application No. 61/547,569, filed on Oct. 14, 2011.

(51) Int. Cl. C12P 7/10

(52) U.S. Cl. CPC C12P 7/10
USPC 435/165; 435/289.1

(57) ABSTRACT

An integrated biological process for cellulosic bioproduct production is provided, which, in one embodiment, results in high ethanol productivity, enzyme recycling and reuse of yeast cells. The integrated process can be an integrated separate hydrolysis and fermentation (SHF) process or an integrated fast simultaneous saccharification and co-fermentation (FSSCF) process. The methods described herein can result in high bioproduct productivity through enzyme recycling and reuse of yeast cells.

### RELATED APPLICATIONS

This application is a continuation under 35 U.S.C. 111(a) of International Application No. PCT/US2012/059898, which application was filed on Oct. 12, 2012 and published in English as WO 2013/106113 on Jul. 18, 2013, which application claims the benefit under 35 U.S.C. 119 (e) of U.S. Provisional Application Ser. No. 61/547,569 (hereinafter the '569 Application) filed on Oct. 14, 2011, U.S. Provisional Application Ser. No. 61/623,408 (hereinafter the '408 Application) filed on Apr. 12, 2012, and U.S. Provisional Application Ser. No. 61/640,536, filed on Apr. 30, 2012, which applications and publications are hereby incorporated by reference in their entireties.

### STATEMENT OF GOVERNMENT RIGHTS

This invention was made with government support under DE-FCO2-07ER64494 awarded by the U.S. Department of Energy. The government has certain rights in the invention.

### BACKGROUND

Current attempts to produce cellulosic-based bioproducts, such as ethanol, are cost prohibitive and involve a number of steps.

### SUMMARY

The embodiments described herein can reduce the process time, and, in various embodiments, can reduce costs. In one embodiment, free living robes (e.g., yeast cells) and/or enzymes are recycled to provide additional benefits.

### BRIEF DESCRIPTION OF THE DRAWINGS

FIG. 1A is a flow diagram of an integrated Separate Hydrolysis and Fermentation (SHF) process according to various embodiments.

FIG. 1B is a flow diagram of an integrated Fast Simultaneous Saccharification and Co-fermentation (FSSCF) process according to various embodiments.

FIGS. 2A and 2B are simplified schematics of tank setups and volume changes for an integrated SHF process (2A) and an integrated FSSCF process (2B) according to various embodiments.

FIGS. 2C and 2D are graphs showing the increase in working volume from cycle to cycle for the integrated SHF Enzyme Hydrolysis (EH) and SHF-fermentation steps, respectively, according to various embodiments.

FIGS. 2E and 2F are graphs showing the increase in working volume from cycle to cycle for the integrated FSSCF-EH and FSSCF-fermentation steps, respectively, according to various embodiments.

FIG. 3 shows and describes an integrated SHF process according to various embodiments.

FIG. 4 shows and describes an integrated FSSCF process according to various embodiments.

FIG. 5A shows the effect of solids loading on the first cycle of an integrated FSSCF process and on the first cycle of an integrated SHF process, comprising a 24 hour (24 h) enzymatic hydrolysis and 24 h SSCF (i.e., FSSCF) or 24 h enzymatic hydrolysis and 24 h hydrolysate fermentation (i.e., integrated SHF) according to various embodiments.

FIG. 5B shows the effect of solids loading on the viable cell density and cell viability after 24 h FSSCF according to various embodiments.

FIG. 6A shows the results of conventional SHF fermentations of AFEX™-CS hydrolysates derived from 6%, 7% and 9% glucan loading enzymatic hydrolysis.

FIG. 6B shows the results of conventional SSCF of AFEX™-CS at 6% and 7% glucan loading.

FIG. 7A-7D show the effect of an initial “inoculation level”, i.e., yeast cell density at 600 nm wavelength (OD₆₀₀) (FIG. 7A), pH (FIG. 7B) and temperature (FIG. 7C) on xylose consumption, ethanol production and cell viability (FIG. 7D) during the first cycle 24 h SSCF of integrated FSSCF at 7% (weight/weight (w/w)) glucan loading according to various embodiments.

FIG. 8 shows optimization results for enzyme loading for cycle 2 during an integrated SHF process according to various embodiments.

FIGS. 9A-9D show integrated SHF process results for sugar concentrations after 24 h enzymatic hydrolysis (FIG. 9A), fermentation performances (FIG. 9B), sugar consumption and ethanol metabolic yield during fermentations (FIG. 9C) and sugar conversions during enzymatic hydrolysis, oligomeric to monomeric sugar conversions during fermentation and overall sugar to ethanol conversions (FIG. 9D).

FIGS. 10A-10D show representative mass balances of processes utilizing 100% glucose/glucan and 100% xylose/xylan, including an integrated SHF process (FIG. 10A), an integrated FSSCF process with enzyme loading “a” (FIG. 10B), an integrated FSSCF process with enzyme loading “b” (FIG. 10C), and an integrated FSSCF process with enzyme loading “c” (FIG. 10D) according to various embodiments.

FIGS. 10E and 10F show mass balances of processes utilizing a commercial protein blend and yeast (i.e., yeast cell) inoculation with AFEX™-Treated Corn Stover in an integrated SHF process (FIG. 10E) and an integrated FSSCF process with enzyme loading “a” (FIG. 10F), according to various embodiments.

FIG. 11 shows conversion (%) during enzyme activity assay for proteins in the removed hydrolysate after enzymatic hydrolysis during an integrated SHF process according to various embodiments.

FIGS. 12A-12C show the effect of enzyme loading profile on ethanol production (FIG. 12A), sugar to ethanol conversions (FIG. 12B), and viable cell density (FIG. 12C) during an integrated FSSCF process according to various embodiments.

FIGS. 13A and 13B show the effect of enzymatic hydrolysis residual solids concentration over time on fermentation in the digested solids (i.e., hydrolysate) according to various embodiments.

FIGS. 14A-14C show comparisons of process ethanol productivity (FIG. 14A) and ethanol yield (FIG. 14B) for integrated SHF and integrated FSSCF processes versus conventional SHF and SSCF processes according to various embodiments.

FIG. 15 is a graph showing optimization of enzyme loading for Cycle 2 of integrated SHF on E-AFEX™ pretreated corn stover according to various embodiments.

FIG. 16 is a graph showing enzymatic hydrolysis for an integrated SHF process on E-AFEX™ pretreated corn stover according to various embodiments.

FIG. 17 is a graph showing fermentation for an integrated SHF process on E-AFEX™ pretreated corn stover according to various embodiments.

FIG. 18 is a graph showing sugar conversion of an integrated SHF process on E-AFEX™ pretreated corn stover according to various embodiments.

FIG. 19 is a graph showing ethanol yield of an integrated SHF process on E-AFEX™ pretreated corn stover according to various embodiments.

FIG. 20 is a graph showing ethanol productivity of an integrated SHF process on E-AFEX™ pretreated corn stover according to various embodiments.

FIG. 21 is a graph showing time scale of an integrated SHF process on E-AFEX™ pretreated corn stover according to various embodiments.

### DETAILED DESCRIPTION

In the following detailed description, the, embodiments are described in sufficient detail to enable those skilled in the art to practice them, and it is to be understood that other embodiments may be utilized and that chemical and procedural changes may be made without departing from the spirit and scope of the present subject matter. The following detailed description is, therefore, not to be taken in a limiting sense, and the scope of the embodiments is defined only by the appended claims.

Processes to produce cellulosic-based ethanol suffer from low ethanol productivity, high enzyme loading and slow xylose fermentation. Other methods attempt to reduce process time by combining enzymatic hydrolysis with fermentation in a “simultaneous” saccharification and co-fermentation (SSCF) process, as this term is understood in the art. However, conventional SSCF processes are quite slow as they require at least 168 h to achieve results comparable to conventional SHF. For example, the enzymatic hydrolysis (EH) step of a conventional Separate Hydrolysis and Fermentation (SHF) process for converting biomass into fermentable sugars can require 96 to 168 hours (h) of process time. It then requires an additional 96 to 168 h to ferment those sugars into ethanol.

In one embodiment, a method is provided comprising performing a first hydrolyzing cycle on lignocellulosic biomass (e.g., pretreated lignocellulosic biomass) containing an easily digestible portion and a non-easily digestible portion, to produce a slurry comprising digested solids and unhydrolyzed solids, respectively; producing a solids residue and a liquid bioproduct from the slurry; recycling the solids residue through a second hydrolyzing cycle wherein an amount of the undigested solids in the solids residue is converted to digested solids; and optionally performing additional hydrolyzing cycles to produce increasing amounts of digested solids which can contain fermentable sugars.

In one embodiment, the method further comprises a separate hydrolysis and fermentation (SHF) step which includes mechanically processing the solids (digested and hydrolyzed) to produce a solids residue and a hydrolysate; and fermenting the hydrolysate to produce a liquid bioproduct which can, in one embodiment, be further processed to yield additional bioproducts.

In one embodiment, the method further comprises performing fast simultaneous saccharifying and co-fermenting (FSSCF) of the slurry to produce a fermentation product; and mechanically processing the fermentation product to produce the solids residue and a liquid bioproduct, which can, in one embodiment, be further processed to yield additional bioproducts. In one embodiment, the process can continue with recycling of the solids residue through a second FSSCF cycle, together with solids (unhydrolyzed and digested) from a second hydrolyzing cycle, to produce a second fermentation product which can be subject to mechanical processing to produce additional solids residue and liquid bioproduct.

The pretreated biomass can be, in one embodiment, an ammonia fiber expansion pretreated biomass, such as AFEX™ pretreated biomass. In one embodiment, the bioproduct is a biofuel, such as ethanol.

In one embodiment, the first easily digestible portion is primarily hemicellulose and, in one embodiment, may further include an amount of cellulose. The non-easily digestible portion can comprise, in one embodiment, primarily lignin, cellulose or combinations thereof, and, in one embodiment, can further comprise an amount of hemicellulose. In one embodiment, the recycled digested solids contain adsorbed enzyme, reusable yeast cells or combinations thereof.

In one embodiment, a system is provided comprising an integrated hydrolysis system configured to perform a first hydrolyzing cycle on lignocellulosic biomass (e.g., pretreated lignocellulosic biomass) containing an easily digestible portion and a non-easily digestible portion to produce a slurry containing digested solids and unhydrolyzed solids, respectively. In one embodiment, the equipment can be further configured to perform a production step in which solids residue and a liquid coproduct are produced from the slurry and recycling of the solids residue through a second hydrolyzing cycle such that an amount of the undigested solids in the solids residue can be converted to digested solids. In one embodiment, additional hydrolyzing cycles can optionally be used to produce increasing amounts of digested solids. The system can further comprise a bioproduct production facility, such as a biofuel production facility, connected to the integrated hydrolysis system and configured to perform either a separate hydrolysis and fermentation (SHF) process or a fast simultaneous saccharification and co-fermentation (FSSCF) process. In one embodiment, the biofuel production facility is an alcohol production facility (e.g., butanol, ethanol, and the like) or a chemical production facility (e.g., lactic acid, butyric acid and the like).

In one embodiment, the system can further include a pretreatment system connected to the integrated hydrolysis system and configured to pretreat lignocellulosic biomass to produce pretreated lignocellulosic biomass. In one embodiment, the system has reduced operating and capital costs as compared to a system relying on a non-integrated hydrolysis system. For example, in one embodiment, the system can comprise fewer fermentation tanks, reduced energy costs, reduced enzyme costs, reduced labor, and so forth, as compared to a system which includes a non-integrated hydrolysis system.

In one embodiment, an apparatus is provided comprising integrated hydrolysis equipment configured to perform a first hydrolyzing cycle on lignocellulosic biomass (e.g., pretreated lignocellulosic biomass) containing an easily digestible portion and a non-easily digestible portion to produce a slurry containing digested solids and unhydrolyzed solids, respectively; a production step in which solids residue and a liquid coproduct are produced from the slurry and recycling of the solids residue through a second hydrolyzing cycle wherein an amount of the undigested solids in the solids residue is converted to digested solids.

In one embodiment, the digested solids comprise fermentable sugars useful in downstream applications as described herein.

In one embodiment, an integrated biological process for cellulosic ethanol production is provided, which results in high ethanol productivity, enzyme recycling and reuse of yeast cells. In one embodiment, the process includes a separate hydrolysis and fermentation (SHF) step or a fast simultaneous saccharification and co-fermentation (FSSCF) step. In one embodiment, this method can result in high ethanol productivity through enzyme recycling and yeast cells reuse. In one embodiment, a high enzymatic hydrolysis rate period is utilized by hydrolyzing an “easily hydrolyzed” portion of lignocellulosic biomass. Additionally, “unhydrolyzed solids,” i.e., the non-easily digestible portion, can be used in various embodiments to bind or adsorb enzymes which are then recycled together with the solids. In one embodiment, the process includes high yeast cell density fermentation and yeast cell recycling. In one embodiment, at least a 17% cost reduction in the production of ethanol from corn stover is achieved as a result of enzyme recycling as compared with a non-integrated process. As such, in one embodiment, the use of enzyme recycling following an AFEX™ pretreatment reduces capital costs, such as by reducing the number of fermentation tanks used to produce a given volume of ethanol by more than 50%.

The term “biomass” as used herein, refers in general to organic matter harvested or collected from a renewable biological resource as a source of energy. The renewable biological resource can include plant materials, animal materials, and/or materials produced biologically. The term “biomass” is not considered to include fossil fuels, which are not renewable.

The term “plant biomass” or “ligno-cellulosic biomass (LCB)” as used herein is intended to refer to virtually any plant-derived organic matter containing cellulose and/or hemicellulose as its primary carbohydrates (woody or non-woody) available for producing energy on a renewable basis. Plant biomass can include, but is not limited to, agricultural crop wastes and residues such as corn stover, wheat straw, rice straw, sugar cane bagasse and the like. Plant biomass further includes, but is not limited to, woody energy crops, wood wastes and residues such as trees, including fruit trees, such as fruit-bearing trees, (e.g., apple trees, orange trees, and the like), softwood forest thinnings, barky wastes, sawdust, paper and pulp industry waste streams, wood fiber, and the like. Additionally grass crops, such as various prairie grasses, including prairie cord grass, switchgrass, big bluestem, little bluestem, side oats grama, and the like, have potential to be produced large-scale as additional plant biomass sources. For urban areas, potential plant biomass feedstock includes yard waste (e.g., grass clippings, leaves, tree clippings, brush, etc.) and vegetable processing waste. Plant biomass is known to be the most prevalent form of carbohydrate available in nature and corn stover is currently the largest source of readily available plant biomass in the United States. When used without a qualifier, the term “biomass” is intended to refer to LCB.

The term “biofuel” as used herein, refers to any renewable solid, liquid or gaseous fuel produced biologically and/or chemically, for example, those derived from biomass. Most biofuels are originally derived from biological processes such as the photosynthesis process and can therefore be considered a solar or chemical energy source. Other biofuels, such as natural polymers (e.g., chitin or certain sources of microbial cellulose), are not synthesized during photosynthesis, but can nonetheless be considered a biofuel because they are biodegradable. There are generally considered to be three types of biofuels derived from biomass synthesized during photosynthesis, namely, agricultural biofuels (defined below), municipal waste biofuels (residential and light commercial garbage or refuse, with most of the recyclable materials such as glass and metal removed) and forestry biofuels (e.g., trees, waste or byproduct streams from wood products, wood fiber, pulp and paper industries). Biofuels produced from biomass not synthesized during photosynthesis include, but are not limited to, those derived from chitin, which is a chemically modified form of cellulose known as an N-acetyl glucosamine polymer. Chitin is a significant component of the waste produced by the aquaculture industry because it comprises the shells of seafood.

The term “agricultural biofuel”, as used herein, refers to a biofuel derived from agricultural crops, lignocellulosic crop residues, grain processing facility wastes (e.g., wheat/oat hulls, corn/bean fines, out-of-specification materials, etc.), livestock production facility waste (e.g., manure, carcasses, etc.), livestock processing facility waste (e.g., undesirable parts, cleansing streams, contaminated materials, etc.), food processing facility waste (e.g., separated waste streams such as grease, fat, stems, shells, intermediate process residue, rinse/cleansing streams, etc.), value-added agricultural facility coproducts (e.g., distiller's wet grain (DWG) and syrup from ethanol production facilities, etc.), and the like. Examples of livestock industries include, but are not limited to, beef, pork, turkey, chicken, egg and dairy facilities. Examples of agricultural crops include, but are not limited to, any type of non-woody plant (e.g., cotton), grains such as corn, wheat, soybeans, sorghum, barley, oats, rye, and the like, herbs (e.g., peanuts), short rotation herbaceous crops such as switchgrass, alfalfa, and so forth.

The term “pretreatment step” as used herein, refers to any step intended to alter native biomass so it can be more efficiently and economically converted to reactive intermediate chemical compounds such as sugars, organic acids, etc., which can then be further processed to a variety of end products such as ethanol, iso-butanol, long chain alkanes etc. Pretreatment can reduce the degree of crystallinity of a polymeric substrate, reduce the interference of lignin with biomass conversion and by hydrolyzing some of the structural carbohydrates, thus increasing their enzymatic digestibility and accelerating the degradation of biomass to useful products. Pretreatment methods can utilize acids of varying concentrations (including sulfuric acids, hydrochloric acids, organic acids, etc.) and/or alkali such as ammonia, ammonium hydroxide, sodium hydroxide, lime, and the like. Pretreatment methods can additionally or alternatively utilize hydrothermal treatments including water, heat, steam or pressurized steam. Pretreatment can occur or be deployed in various types of containers, reactors, pipes, flow through cells and the like. Most pretreatment methods will cause the partial or full solubilzation and/or destabilization of lignin and/or hydrolysis of hemicellulose to pentose sugars.

The term “moisture content” as used herein, refers to the quantity of water in biomass. Moisture content is usually expressed on a dry weight basis (dwb) as follows: MC_{dwb}={(Wᵢ−W_{f})/W_{f}}*100, wherein Wᵢ is the initial weight of biomass before drying and W_{f} is the final weight of biomass after drying.

The term “Ammonia Fiber Expansion” (hereinafter “AFEX™”) pretreatment” as used herein, refers to a process for pretreating biomass with ammonia to solubilize lignin from plant cell wall and redeposit to the surface of the biomass. An AFEX™ pretreatment disrupts the lignocellulosic matrix, thus modifying the structure of lignin, partially hydrolyzing hemicellulose, and increasing the accessibility of cellulose and the remaining hemicellulose to subsequent enzymatic degradation. Lignin is a primary impediment to enzymatic hydrolysis of native biomass, and removal or transformation of lignin is a suspected mechanism of several of the leading pretreatment technologies, including AFEX™.

However, in contrast to many other pretreatments, the lower temperatures and non-acidic conditions of the AFEX™ process inhibits lignin from being converted into furfural, hydroxymethyl furfural, and organic acids that can negatively affect microbial activity. The process further expands and swells cellulose fibers and further breaks up amorphous hemicellulose in lignocellulosic biomass. These structural changes open up the plant cell wall structure, thus enabling more efficient and complete conversion of lignocellulosic biomass to value-added products, while preserving most, up to all, of the nutrient value and most, up to all, of the original composition of the material. See, for example, the methods described in U.S. Pat. Nos. 6,106,888; 7,187,176; 5,037,663 and 4,600,590, all of which are hereby incorporated by reference herein in their entireties.

The term “hydrolysis process” or “hydrolysis” as used herein, refers to a process in which lignocellulosic biomass is hydrolyzed, i.e., broken down or cleaved into its individual constituent sugars. Such hydrolysis can be performed using an enzyme or acid treatment.

The term “integrated hydrolysis” as used herein, refers to a hydrolysis process in which biomass is recycled through the process, i.e., the biomass is subject to more than one cycle of the hydrolysis process. This is in contrast to “non-integrated hydrolysis” which does not include any recycle steps.

The term “easily digestible portion” as used herein, refers to a solids portion of lignocellulosic biomass which can be hydrolyzed into individual constituent sugars after being subjected to a single cycle of a hydrolysis process.

The term “non-easily digestible portion” as used herein, refers to a solids portion of lignocellulosic biomass which is not hydrolyzed after a single cycle of a hydrolysis process.

The term “cycle” as used herein refers to an event in a hydrolysis process in which an amount of an unhydrolyzed solids portion of lignocellulosic biomass and an amount of enzymes are newly combined and subjected to hydrolysis.

The term “Fast SSCF” or “FSSCF” as used herein, refers to a SSCF process which is performed in less than 24 h.

Cellulosic biofuel production from lignocellulosic biomass has gained considerable momentum due to both environmental and social sustainability benefits. However, the technology is not yet fully commercialized. One issue impeding cellulosic biofuel production using the sugar platform is the hydrolysis-resistant nature of certain components in the lignocellulosic biomass.

Nearly all forms of lignocellulosic biomass, i.e., plant biomass, such as monocots, comprise three primary chemical fractions: hemicellulose, cellulose, and lignin. Lignin which is a polymer of phenolic molecules, provides structural integrity to plants, and is difficult to hydrolyze. As such, after sugars in the biomass have been fermented to a bioproduct, such as alcohol, lignin remains as residual material, i.e., a non-easily digestible portion.

Cellulose and hemicelluloses in plant cell walls exist in complex structures within the residual material. Hemicellulose is a polymer of short, highly-branched chains of mostly five-carbon pentose sugars (xylose and arabinose), and to a lesser extent six-carbon hexose sugars (galactose, glucose and mannose). Because of its branched structure, hemicellulose is amorphous and relatively easy to hydrolyze into its individual constituent sugars by enzyme or dilute acid treatment. Cellulose is a linear polymer comprising of β (1→4) linked D-glucose in plant cell wall, much like starch with a linear/branched polymer comprising of α (1→4) linked D-glucose, which is the primary substrate of corn grain in dry grain and wet mill ethanol plants. However, unlike starch, the glucose sugars of cellulose are strung together by β-glycosidic linkages which allow cellulose to form closely-associated linear chains. Because of the high degree of hydrogen bonding that can occur between cellulose chains, cellulose forms a rigid crystalline structure that is highly stable and much more resistant to hydrolysis by chemical or enzymatic attack than starch or hemicellulose polymers. Although hemicellulose sugars represent the “low-hanging” fruit for conversion to a biofuel, the substantially higher content of cellulose represents the greater potential for maximizing biofuel yields, on a per ton basis of plant biomass.

Therefore, a pretreatment process is typically used to alter and open up the cell wall matrix, to hydrolyze the hemicelluloses, and to reduce crystallinity. Pretreatment disrupts the non-easily digestible portion of lignocellulosic biomass, e.g., cellulose and lignin, thus improving its digestibility. After pretreatment, much of the biomass becomes easily digestible while a c a portion, remains non-easily digestible. Ultimately, the pretreatment process makes the cellulose more accessible (during a subsequent hydrolysis process) for conversion of the carbohydrate polymer into fermentable sugars. Ammonia fiber expansion (AFEX™), for example, is capable of opening up the cell wall in agricultural residues with greatly reduced degradation products compared to acidic pretreatments.

Other pretreatment methods include, for example, ammonia recycled percolation (ARP), concentrated acid hydrolysis pretreatment, dilute acid hydrolysis, two-stage acid hydrolysis pretreatment, high pressure hot water-based methods, i.e., hydrothermal treatments such as steam explosion and aqueous hot water extraction, reactor systems (e.g., batch, continuous flow, counter-flow, flow-through, and the like), lime treatment and a pH-based treatment, hydrothermal or chemical pretreatments, followed by an enzymatic hydrolysis (i.e., enzyme-catalyzed hydrolysis) or simultaneous enzymatic hydrolysis and saccharification. As noted above, some methods generate nearly complete hydrolysis of the hemicellulose fraction for efficient recovery of high yields of the soluble pentose sugars. Recovery of these sugars also facilitates the physical removal of the surrounding hemicellulose and lignin, thus exposing the cellulose to later processing.

However, the enzyme loadings for such hydrolysis can be high. For example, typical enzyme loadings for conventional (non-integrated) enzymatic hydrolysis of corn stover can require at least 30 up to 36 mg protein per gram glucan. According to the National Renewable Energy Laboratory's (NREL, Golden Colorado) 2011 report, enzyme costs can account for as much as 15.7% of the total hydrolysis costs, even if enzyme loading is 20 mg per gram glucan.

The fastest (i.e., highest) hydrolysis rates are typically seen during an initial time period, such as during the first 24 h of an enzymatic hydrolysis process, during which the easily digestible portion of biomass are being hydrolyzed. Thereafter, the non-easily digestible portion of the biomass are hydrolyzed, but at a slower (i.e., lower) rate. The hydrolysis rate can also be slower at this stage due to the presence of product inhibition, enzymes inactivation and thermal instability of cellulases. Reduced hydrolysis rates not only can lead to longer enzymatic hydrolysis times, but also rely on higher enzyme loadings as discussed above.

As such, an additional impediment to commercialization is the slow speed of xylose fermentation. For example, a conventional separate hydrolysis and fermentation (SHF) process for corn stover can take at least 72 up to about 168 h to achieve high sugar conversions at high solids loading and another 96 to 168 h to ferment those sugars (mostly glucose and xylose) into ethanol. Thus, overall biological processing times can be well over 5 days, leading to even higher capital and operating costs.

Xylose can be co-fermented with glucose to ethanol by genetically modified strains such as Escherichia coli KO11, Zymomonas mobilis AX101 and Saccharomyces cerevisiae 424A(LNH-ST) or using native strains like Pichia stipites, candida, native Saccharomyces cerevisiae. Performance of those strains in AFEX™ (ammonia fiber expansion) pretreated hydrolysate was good, with satisfactory hydrolysate fermentation. However, S. cerevisiae 424A(LNH-ST) fermented xylose slowly in AFEX™ hydrolysates. Slow xylose fermentation by this strain might be caused by redox imbalance, no specialized xylose transporter, and degradation products inhibition. Degradation products mostly inhibit the yeast cell growth during fermentation and hence reduce the overall xylose fermentation rate. Due to the slow xylose consumption, the fermentation ethanol productivity (ethanol produced per unit of time) is generally much lower than the industrial criterion of about 1.0 g·L⁻¹·h⁻¹.

As such, an additional issue impacting the commercialization of cellulosic-based biofuels is the low production rate (e.g., ethanol production rate) achievable, such as when using a conventional SHF process.

In one embodiment, an integrated hydrolysis process is provided which can include recycling of biomass, enzymes and yeast in one or more recycling steps. In one embodiment, pretreated lignocellulosic biomass (hereinafter “biomass”) containing an easily digestible portion (as a result of, for example, pretreatment) and a non-easily digestible portion, which can be subjected to a first cycle of a hydrolysis process to hydrolyze substantially all of the easily digestible portion into digested solids (which can include, for example, hydrolyzed sugars). While the non-easily digestible portion may not be hydrolyzed initially, such as after one cycle, in one embodiment, exposure to the hydrolysis process for a sufficient period of time and/or for a sufficient number of cycles can cause at least an amount of the non-easily digestible portion to become an easily digestible portion in a subsequent cycle.

In one embodiment, the product (now containing digested solids and a new easily digestible portion of biomass) from the first cycle of hydrolysis is again subjected to hydrolysis. In one embodiment, at least a portion the new easily digestible portion is hydrolyzed after the second cycle of hydrolysis. Any number of hydrolysis cycles may be used until most, up to substantially all of the biomass is hydrolyzed. In one embodiment, substantially all the new easily digestible portion produced after the first cycle is substantially hydrolyzed after the second cycle.

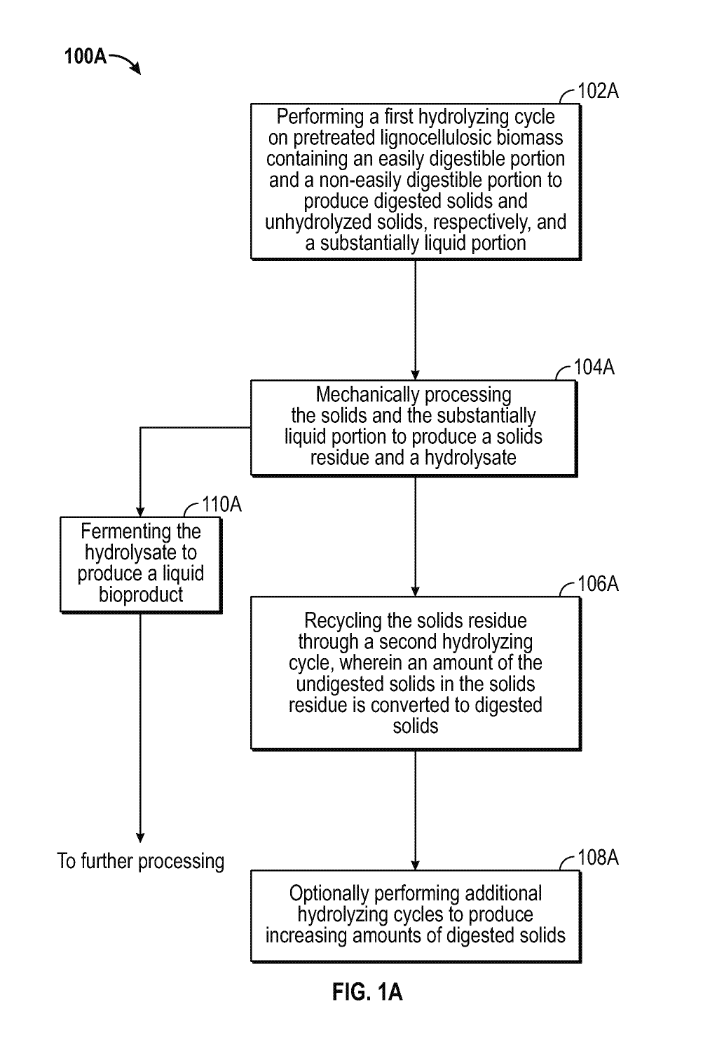

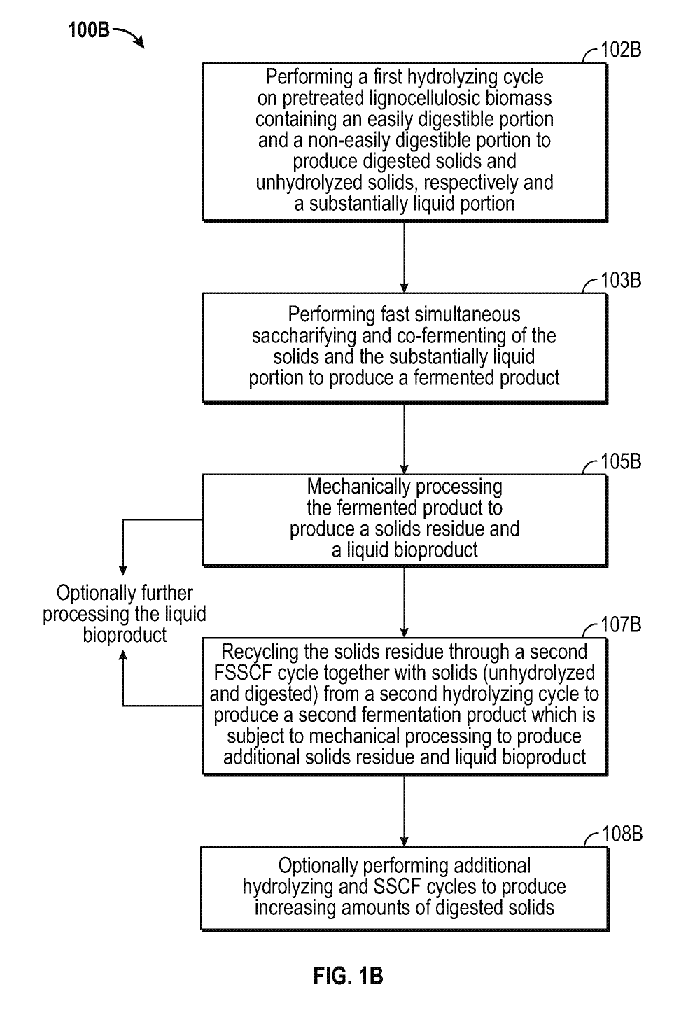

In the embodiment shown in FIG. 1A, an integrated SHF process 100A is provided comprising performing 102A a first hydrolyzing cycle on pretreated lignocellulosic biomass containing a first easily digestible portion and a non-easily digestible portion to produce a slurry containing digested solids and unhydrolyzed solids, respectively. Thereafter, the process comprises mechanically processing 104A the slurry to produce a solids residue and a hydrolysate, and recycling 106A the solids residue through a second hydrolyzing cycle, wherein an amount of the undigested solids in the solids residue is converted to digested solids.

In one embodiment, the process includes fermenting 110A the hydrolysate produced in step 104A to produce a liquid bioproduct which can be subjected to further processing to produce bioproducts such as biofuels (e.g., bio-alcohols such as ethanol and butanol), chemical intermediates for biochemical synthesis of bioproducts (e.g., organic acids, fatty acids, alkanes, terpenoids, and the like) and the like.

The process 100A can further include optionally performing 108A additional hydrolyzing cycles to produce increasing amounts of digested solids. In one embodiment, the digested solids comprise digested solids containing fermentable sugars. In one embodiment, the process additionally or alternatively includes optionally further processing an amount of the digested solids, such as from steps 106A and 108A, for use in bioproducts such as bio-alcohols and chemical intermediates as noted above, further including, for example, biocomposites, which are materials formed by a matrix (resin) and a reinforcement of natural fibers typically derived from plants or cellulose.

In the embodiment shown in FIG. 1B, an integrated FSSCF process 100B is provided, which comprises performing 102B a first hydrolyzing cycle on pretreated lignocellulosic biomass containing a first easily digestible portion and a non-easily digestible portion to produce a slurry containing digested solids and unhydrolyzed, solids, respectively. Thereafter, the process 100B comprises performing 103B simultaneous saccharifying and co-fermenting of the slurry to produce a fermentation product, and mechanically processing 105B the fermentation product to produce a solids residue and a liquid bioproduct. Thereafter, the process continues with recycling 107B substantially all the solids residue through a second SSCF cycle together with substantially all the solids (unhydrolyzed and digested) from a second hydrolyzing cycle to produce a second fermentation product which can be subject to mechanical processing to produce additional solids residue and liquid bioproduct. In one embodiment (not shown), only a portion of the solids residue is recycled through the second SSCF cycle together with a portion or substantially all the solids from the second hydrolyzing cycle. In one embodiment (not shown), substantially all the solids residue is recycled through the second SSCF cycle together with a portion of the solids from the second hydrolyzing cycle.

In one embodiment, the process includes optionally further processing at least a portion of the liquid bioproduct, such as from steps 105B and 107B, to produce bioproducts, such as biofuels (e.g., bio-alcohols such as ethanol and butanol), chemical intermediates for biochemical synthesis of bioproducts (e.g., organic acids, fatty acids, alkanes, terpenoids, etc.) and the like.

The process 100B can further comprise optionally performing 108B additional hydrolyzing cycles to produce increasing amounts of digested solids. In one embodiment, the digested solids comprise digested solids containing fermentable sugars. In one embodiment, the process additionally or alternatively includes optionally further processing an amount of the digested solids, such as from steps 107B and 108B, for use in bioproducts such as bio-alcohols and chemical intermediates as noted above, further including, for example, biocomposites.

In one embodiment, including in the processes of 100A and/or 100B (FIGS. 1A and/or 1B) the hydrolysis is an enzyme hydrolysis and the digested solids are predominantly comprised of hydrolyzed solids, such as hydrolyzed sugars, which can include, for example, monomeric sugars (i.e., glucose, xylose, arabinose, mannose and/or galactose) and/or oligomeric sugars such as gluco-oligomers (i.e., linear chains of glucose units with varying degrees of polymerization (DP)) and/or xylo-oligomeric sugars (i.e., xylose backbone polysaccharides with branching (linear or with varying DP) from other sugars).

Any suitable type of lignocellulosic biomass can be used. In one embodiment, the easily digestible portion is hemicellulose and the non-easily digestible portion includes lignin and/or cellulose. In one embodiment, the lignocellulosic biomass is selected from corn stover (CS), switchgrass, rice straw and combinations thereof.

Any suitable type of pretreatment can be used prior to the integrated hydrolysis step. In one embodiment, an ammonia pretreatment process such as AFEX™ is used. AFEX™ is a “dry to dry” process such that biomass enters and exits the process in the same “no free water” state. In one embodiment, corn stover is subjected to an AFEX™ pretreatment, referred to herein as AFEX™-CS. In one embodiment, the AFEX™-CS is not subjected to washing, detoxification and/or nutrient supplementation in subsequent hydrolysis/fermentation steps.

In one embodiment, Extractive Ammonia Fiber Expansion (E-AFEX™) pretreatment is used. E-AFEX™ convents cellulose I to cellulose III and removes a portion of the lignin in the biomass. As a result, in one embodiment, E-AFEX™ pretreated biomass can have a higher enzyme digestibility as compared to other variations of AFEX™ pretreatments.

In one embodiment, the hydrolysis is enzyme hydrolysis. While not wishing to be bound by this proposed theory, it is possible that acid pretreated biomass and other pretreated biomass may also work in the processes described herein.

In one embodiment, AFEX™-CS can be hydrolyzed using commercial enzymes and S. cerevisiae. In one embodiment, a more heat tolerant microbial strain can be used, such as Kluyveromyces marxianus or Thermoanaerobacterium saccharolyticum. The easily digestible portion can, in various embodiments, be hydrolyzed first and within 24 h, thus avoiding slower hydrolysis rates which typically occur after 24 h.

In one embodiment, the remaining unhydrolyzed solids, now containing an amount of adsorbed enzymes, can be recycled for further hydrolysis, i.e., provided to a second cycle of hydrolysis. The second (as well as subsequent hydrolysis cycles) can be performed in sequential tanks and/or recycled into the same tank. By recycling the unhydrolyzed solids, an amount of the enzyme is also recycled, thus reducing enzyme loading requirements as compared with a non-integrated hydrolysis process.

In one embodiment, enzyme loading can be reduced by at least 1% up to about 10% or higher, such as up to about 15%, 20%, 25% or higher such as up to about 28% or higher, such as up to about 39% or higher, including any range there between. In one embodiment enzyme loading is reduced by at least about 25%, such as at least 27.7%. In one embodiment, enzyme loading is reduced by at least about 40% such as at least 38.9%. In a particular embodiment, using corn stover, enzyme loading is reduced from no less than 36 mg protein per g glucan down to no more than 22.3 mg or 25.8 mg protein per g glucan. In one embodiment, enzyme loading is reduced further, such as down to 20 mg/g or lower, such as down to 15 mg/g or lower, such as down to 10 mg/g or lower, such as down to 5 mg/g or lower, including any range there between, such as by changing the enzyme type and/or proportions.

In one embodiment, fermentation of xylose is completed more quickly as compared to fermentation following a non-integrated hydrolysis processes. In one embodiment, fermentation can be completed in less than 24 h.

In one embodiment, the integrated process utilizes a high yeast inoculation level as compared to a conventional non-integrated hydrolysis process (with no recycle steps), such as at least 10 times higher up to about 40 times higher, including any range there between. In a particular embodiment, inoculation level (OD) is at least 20 as compared to 0.5 to 2 for a non-integrated hydrolysis process. In one embodiment, fast xylose fermentation, i.e., fermentation in less than 24 h, was achieved using a higher inoculation level, i.e., greater than 2 OD.

In one embodiment, high inoculation level fermentation bypasses the degradation products inhibition on yeast cell growth and improves xylose fermentation. In one embodiment, the integrated process includes an optional yeast cell recycling step. In one embodiment, the enzymatic hydrolysis takes 24 h or less and/or one cycle of fermentation step takes 24 h or less. The substantially liquid portion of the hydrolysis/fermentation mixture can then be harvested and subjected to distillation. In one embodiment, unhydrolyzed solids can be repeatedly transferred to the next hydrolysis/fermentation cycle to allow more hydrolysis time. In one embodiment, up to five cycles of hydrolysis/fermentation are used, with an increasing amount of digested solids produced with each hydrolysis cycle. Since some enzymes can be recycled by this approach and part of the inhibitory degradation products contained in the substantially liquid portion can be removed during the subsequent cycles, the hydrolysis of the residual solids can be both more rapid and more complete (higher sugar conversions). In one embodiment, no enzymes are added during the final cycle of enzymatic hydrolysis. In other embodiments, additional biomass can be added to the subsequent hydrolysis cycles, as well as additional water and/or enzymes, as desired.

Additionally, the novel integrated hydrolysis processes described herein can also improve bioproduct productivity. In one embodiment, the bioproduct is a biofuel such as alcohol. In one embodiment, the alcohol is ethanol. In a particular embodiment, process ethanol productivity can be enhanced by at least two fold as compared to a non-integrated hydrolysis process. As a result of the increased speed of both hydrolysis and fermentation, in one embodiment, the increase is between about 2 and 3 fold, including any range there between.

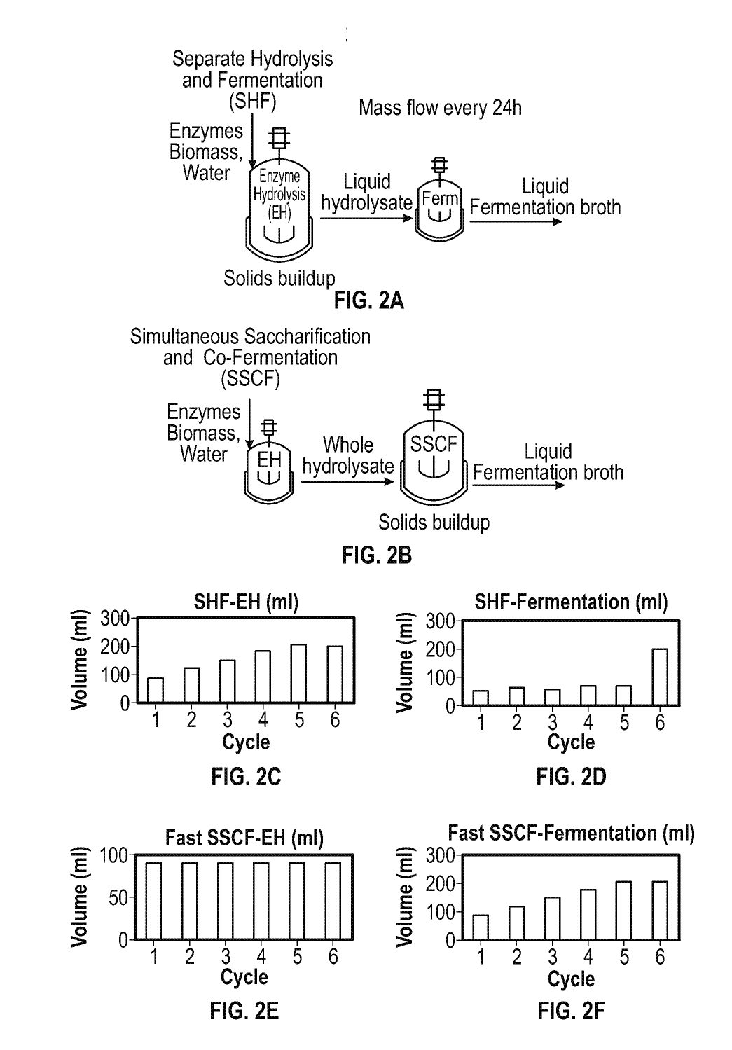

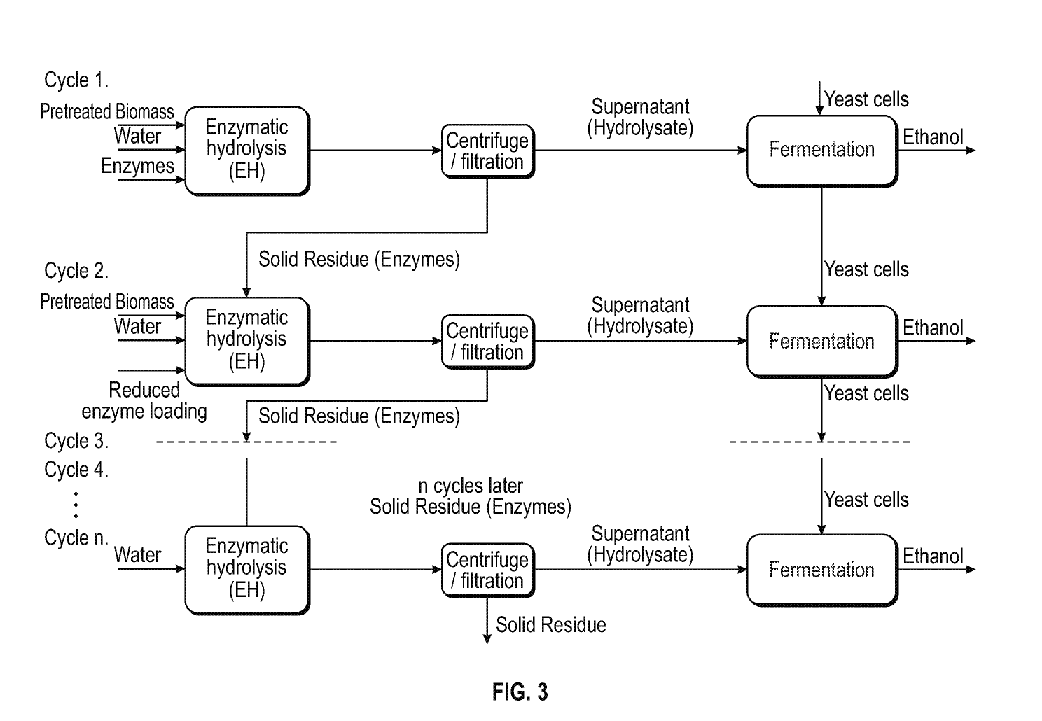

In one embodiment, the process is an integrated separate hydrolysis and fermentation (SHF) process as shown in FIG. 2A. One example of such a process is discussed in Example 1 and shown in FIG. 3.

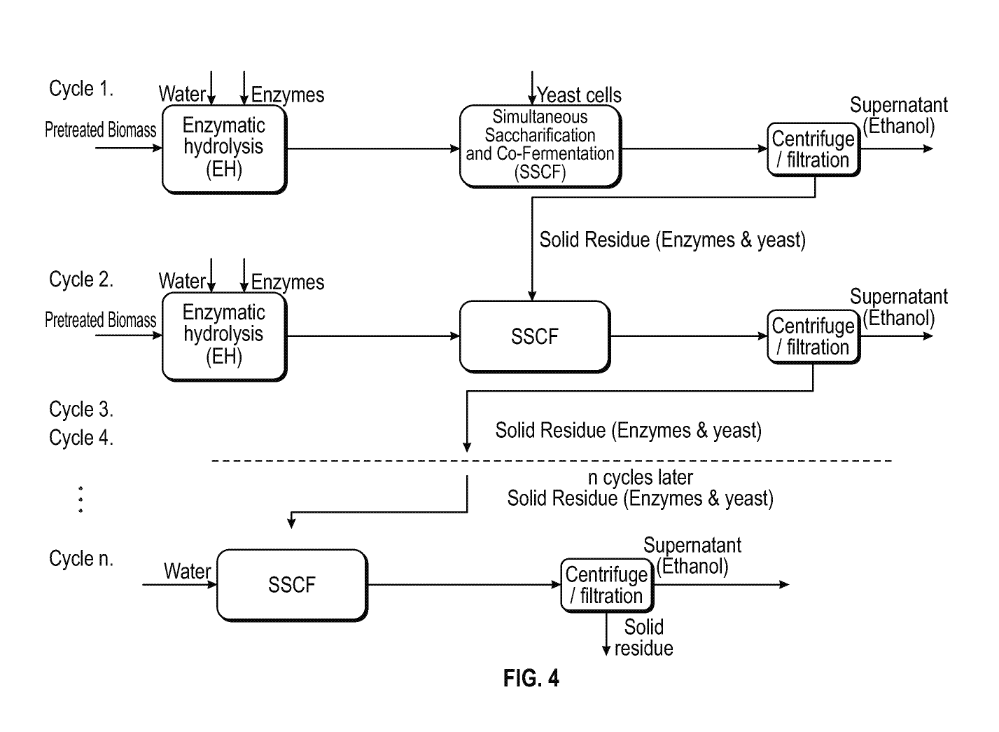

In one embodiment, the process is an integrated fast saccharification and co-fermentation (FSSCF) process as shown in FIG. 3B. One example of such a process is discussed in Example 1 and shown in FIG. 4.

In one embodiment, alcohol production, such as ethanol or butanol production is increased by using the methods described herein. In one embodiment, the integrated SHF process and FSSCF processes described herein save time by quickly fermenting xylose, processing the easily digestible biomass first to fully utilize the high enzymatic hydrolysis rate period, and hydrolyzing the non-easily digestible portion of biomass in the presence of less inhibition by degradation products and sugars, to enhance ethanol productivity.

In one embodiment, ethanol productivity is at least three times higher, and in some embodiments, more than three times higher than ethanol productivity achieved using a conventional SHF and SSCF process.

In one embodiment, a novel integrated biological process is provided. The easily hydrolyzed portion of biomass can be converted to ethanol first, which, in one embodiment, is completed in no more than about 48 h (24 h enzyme hydrolysis plus 24 h fermentation). In one embodiment, the liquid can then be harvested and subjected to distillation. In one embodiment, the less easily hydrolyzed portion, which carries an amount of enzyme and yeast cells, can then be recycled through the hydrolysis process again. In this way, enzymes and yeast cells are largely reused.

In one embodiment, at least two, up to three or four cycles are completed. In one embodiment, five cycles are used to substantially hydrolyze the most non-easily digestible portion of the biomass. Since enzymes are accumulated to a relatively high concentration in the unhydrolyzed solids, and part of the decomposition products are removed with liquid during the five cycles, in one embodiment the hydrolysis more easily achieves high sugar conversion. For cost-effective production of cellulosic ethanol, the ethanol concentration can be, in one embodiment, above 40 g/L and fermentation productivity can be, in one embodiment, above 1 g/L/h. In one embodiment ethanol productivity can be calculated based on enzyme hydrolysis time plus fermentation time (process ethanol productivity).

In one embodiment, the integrated process described herein results in a cost reduction for the production of cellulosic biofuel of more than 1% up to about 18%. In one embodiment, the cost reduction for production of cellulosic biofuel is at least 18%.

Embodiments will be further described by reference to the following examples, which are offered to further illustrate various embodiments of the present subject matter. It should be understood, however, that many variations and modifications may be made while remaining within the scope of the present subject matter.

### Example 1

### AFEX™ Pretreated Corn Stover (AFEX™-CS)

AFEX™-CS with glucan and xylan contents of 34.1% and 20.4%, respectively, from GLBRC (Great Lakes Bioenergy Research Center) was used. AFEX™ pretreatment was conducted in a 2 L Parr high pressure stainless steel reactor vessel with temperature and pressure probes attached according to the method described in V. Balan, et al., in Biofuels:Methods and protocols, Methods in Molecular Biology, 2010, pp. 61-77, which is hereby incorporated by reference herein in its entirety. AFEX™ pretreatment conditions were as follows: ammonia to biomass loading 1.0 g/g dry biomass, water loading 0.6 g/g dry biomass, temperature 140° C. and residence time 15 minutes.

### Microorganism

The xylose-fermenting strain Saccharomyces cerevisiae 424A(LNH-ST) was provided by Purdue University. LNH-ST is a genetically modified strain with multiple copies of xylose reductase (XR) and xylitol dehydrogenase (XD) genes from Scheffersomyces (Pichia) stipitis and an endogenous xylulokinase gene (XK) incorporated in the chromosome. The seed culture of this strain was prepared in a 250 ml Erlenmeyer flask with 100 ml YPDX medium (10 g/L yeast extract, 20 g/L peptone, 75 g/L glucose and 20 g/L xylose). A frozen glycerol stock was used for seed culture inoculation. As all inoculation levels in each example described herein were performed at a wavelength of 600 nm. As such, use of the term “OD,” including in the Figures, is intended herein to refer to “OD₆₀₀.” The initial inoculation level (OD₆₀₀) was approximately 0.1. Seeds were cultured at 30° C. and 150 rpm under micro-aerobic conditions for 24 hrs.

### Enzymatic Hydrolysis

For enzymatic hydrolysis (EH) in both the integrated SHF and FSSCF processes described below, the inputs for each cycle, including fresh AFEX™-CS, water, phosphate buffer, and enzymes were 100 g in total. Glucan loadings for integrated SHF and FSSCF processes were 7.5% and 7% (w/w), respectively, which correspond to 22.0 g and 20.5 g AFEX™-CS biomass in 100 g total mixture, respectively.

Enzymes used for the hydrolysis included Accellerase 1500 (cellulase), Accellerase XY (xylanase), and Multifect pectinase (Genencor Inc., USA), with protein loadings of 30, 3, and 3 mg/g glucan, respectively, in the first cycle. The protein loadings were varied during cycles 2 through 5. Enzymatic hydrolysis was performed at 50° C., pH 4.8, and 250 rpm. Fifty mg/L chloramphenicol (Cm) was used during hydrolysis to avoid microbial contamination.

### Integrated SHF Process

Enzymatic hydrolysis was performed on the AFEX™-CS via an integrated SHF process for 24 h, followed by centrifugation at 5300 rpm for 20 minutes as shown in FIG. 2. The resulting unhydrolyzed solids were recycled to the next enzymatic hydrolysis tank, thus increasing the working volume from cycle to cycle for the enzyme hydrolysis (EH) step as shown in FIG. 2C. Ethanol fermentation of the liquid hydrolysate was conducted in a 250 ml unbaffled flask at 150 rpm, 32° C. and pH 5.5 with an initial OD₆₀₀ of 20 for 24 h. The last step (Cycle 6) fermentation was conducted in the last EH tank after a 24 h hydrolysis. Therefore, the last step fermentation has the same working volume as the last EH (FIG. 2D).

After fermentation, yeast cells were collected by centrifugation at 4400 rpm for 8 minutes and used for the next fermentation inoculation. As shown in FIG. 3, solids residue and yeast cells were continually recycled to the next round of enzymatic hydrolysis and fermentation, respectively. For enzymatic hydrolysis in the last step, 5 ml phosphate buffer and 55 ml water were added without any new enzymes or fresh biomass. Enzymatic hydrolysis for cycles 1, 2, 3 was conducted in a 250 ml baffled flask while cycles 4, 5 and the last step were performed in a 500 ml baffled flask. See FIG. 3 for details.

### Integrated FSSCF Process

Enzymatic hydrolysis was performed on the AFEX™-CS via an integrated FSSCF process for 24 h, followed by a change of conditions to pH 5.5, 32° C., and 180 rpm, and an inoculation level (initial OD₆₀₀=20) (FIG. 4). After 24 h of FSSCF, centrifugation was carried out at 5300 rpm for 20 minutes. Thereafter, the hydrolysate was distilled. The solids were then recycled and transferred to the next cycle SSCF tank, thus increasing the working volume from cycle to cycle for the integrated FSSCF-SSCF step as shown in FIGS. 2E and 2F, respectively. After 5 cycles, 5 ml phosphate buffer and 55 ml water were added to the solids residue with no enzymes and no fresh biomass supplemented for the final cycle. Ethanol fermentation of the liquid hydrolysate was conducted in a 250 ml baffled flask at 32° C., pH 5.5 and 180 rpm for 72 h.

The last step is denoted as cycle 6 in FIGS. 2C-2F. Basically, a volume increase occurred in the EH tank for the integrated SHF process and in the SSCF tank for the integrated FSSCF process due to the buildup of solids residue. The last fermentation step for the integrated SHF process was carried out in the EH tank after 24 h hydrolysis without solid-liquid separation. See FIG. 4, which shows and describes the process used. After the last step of FSSCF, the solids and liquid can be separated before or after ethanol distillation. The solids can be burned for producing electricity and/or heat.

### Conventional SHF and SSCF Processes

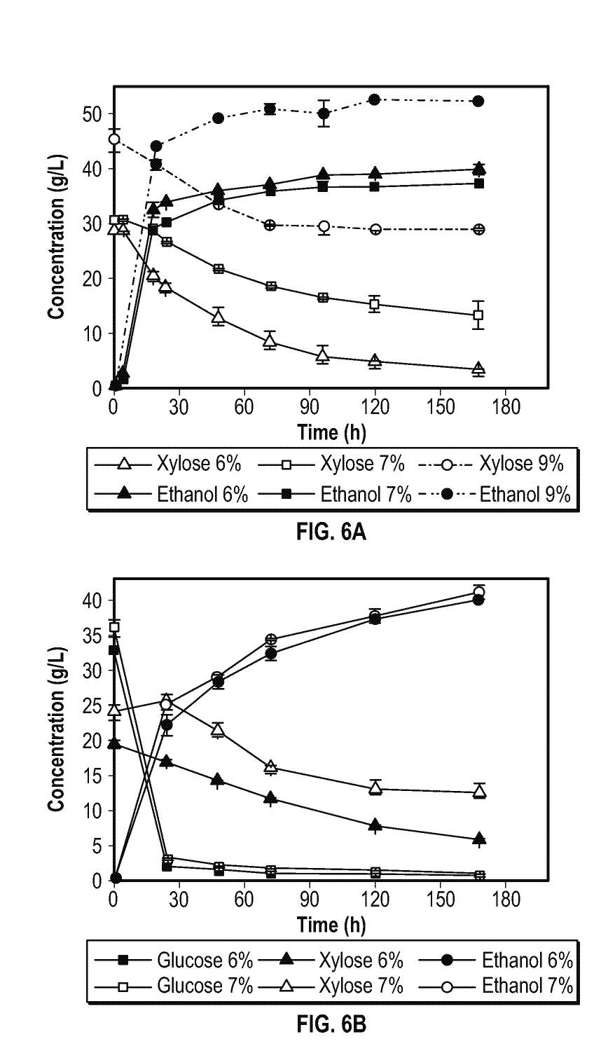

For comparison purposes, enzymatic hydrolysis was also performed on the AFEX™-CS via a conventional SHF process at 50° C., pH 4.8 and 250 rpm for 96 h. The resulting hydrolysate was used for fermentation at 30° C., pH 5.5 and 150 rpm with an initial inoculation level (OD₆₀₀) of 2.0. A conventional SSCF process was carried out at 50° C., pH 4.8 and 250 rpm for 6 h. The conditions were then changed to 30° C., pH 5.5 and 180 rpm, with yeast cells inoculated at an initial OD₆₀₀ of 2.0. For purposes of comparison, the enzyme loading used for the conventional processes was the same as was used in the first cycle of the integrated processes tested herein. (See FIGS. 6A and 6B).

### Measurement of Viable Cells Density

Viable cell density was measured in colony forming unit (CFU) per ml. The samples were first diluted 25,000 times. Then 20 μl of a diluted sample was plated on YPD agar medium (5 g/L yeast extract, 10 g/L tryptone, 25 g/L glucose, 20 g/L agar and 50 mg/L chloramphenicol). Single colonies were counted after the plate was incubated for 24 h at 30° C. and viable cell density was calculated accordingly.

### HPLC Analysis and Mass Balance

Glucose, xylose and ethanol concentrations were analyzed by HPLC using a Biorad Aminex HPX-87H column according to known methods. Column temperature was maintained at 50° C. Mobile phase (5 mM H₂SO₄) flow rate was 0.6 mL/min.

Mass balances and consumed sugars were calculated based on the analysis of monomeric, oligomeric and polymeric sugars in samples as described herein. Ethanol metabolic yield was calculated based on the theoretical ethanol yield from consumed glucose and xylose, which is 0.51 g ethanol/g glucose or xylose. Process ethanol productivity was calculated based on enzymatic hydrolysis time plus fermentation time.

### Results and Discussion

### Optimization of Fermentation Via Adjustment of Solids Loading

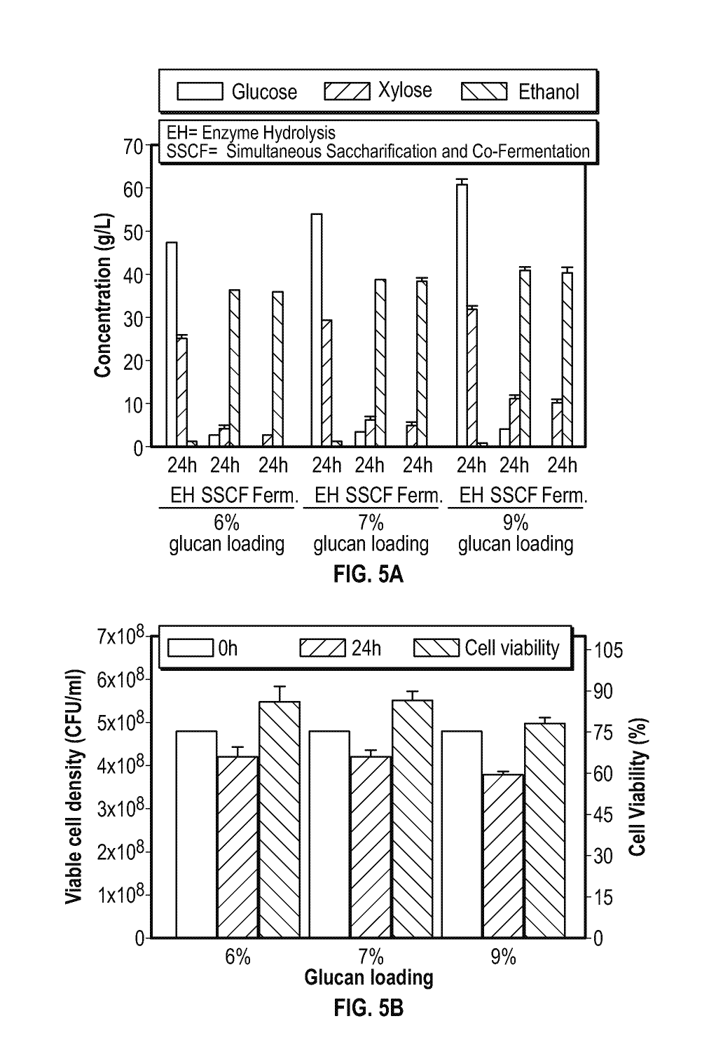

The effect of solids loading (6%, 7% and 9% glucan loading) was investigated by performing 24 h enzymatic hydrolysis, followed by 24 h SSCF (the first cycle of an integrated FSSCF process) and by performing 24 h enzymatic hydrolysis followed by 24 h hydrolysate fermentation (the first cycle of an integrated SHF process) (FIG. 5A). One objective was to produce ethanol at an industrially relevant titer of 40 g/L or higher.

The ethanol titer of 40 g/L was achieved with a 9% glucan loading by both the integrated FSSCF and the integrated SHF processes. However, the remaining xylose concentration in the fermentation broth was almost two times higher than the 7% glucan loading. Since the broth was removed and subjected to distillation after 24 h fermentation (FIG. 3 and FIG. 4), this result suggests a greater loss of fermentable sugars as the glucan loading increases, such as the 9% glucan loading. Moreover, the cell viability during FSSCF at the higher glucan loading (9%) was lower as compared to lower solids loadings (FIG. 5B). This result suggests that yeast cells were dying more rapidly at the higher glucan loading of 9%, and thus fewer viable yeast cells were available for recycle in subsequent cycles. Although still a viable option, the relatively slower xylose fermentation and reduced yeast cell viability at loading at (and likely above) 9% glucan loading appears to be the result of a higher concentration of biomass degradation products. (See also FIGS. 6A and 6B which shows a similar trend for conventional SHF and SSCF processes).

Lower glucan loadings, such as no more than about 7% appear to improve performance. As can be seen with the results herein, a glucan loading of 7% produced 38.6 g/L ethanol with 6.0 g/L xylose remaining in the broth after 24 h hydrolysis and 24 h SSCF (first cycle of the integrated FSSCF) (FIG. 5A).

As discussed below, similar results were obtained at 7% glucan loading with an integrated separate hydrolysis and fermentation (SHF) process, i.e., a 24 h hydrolysis followed by a 24 h hydrolysate fermentation process, which yielded 38.1 g/L ethanol with 4.8 g/L xylose remaining unutilized (FIG. 5A). As such, in one embodiment, by converting 4 g/L more xylose to ethanol, at least 40 g/L ethanol can be achieved.

### Further Optimization of Fermentation Conditions

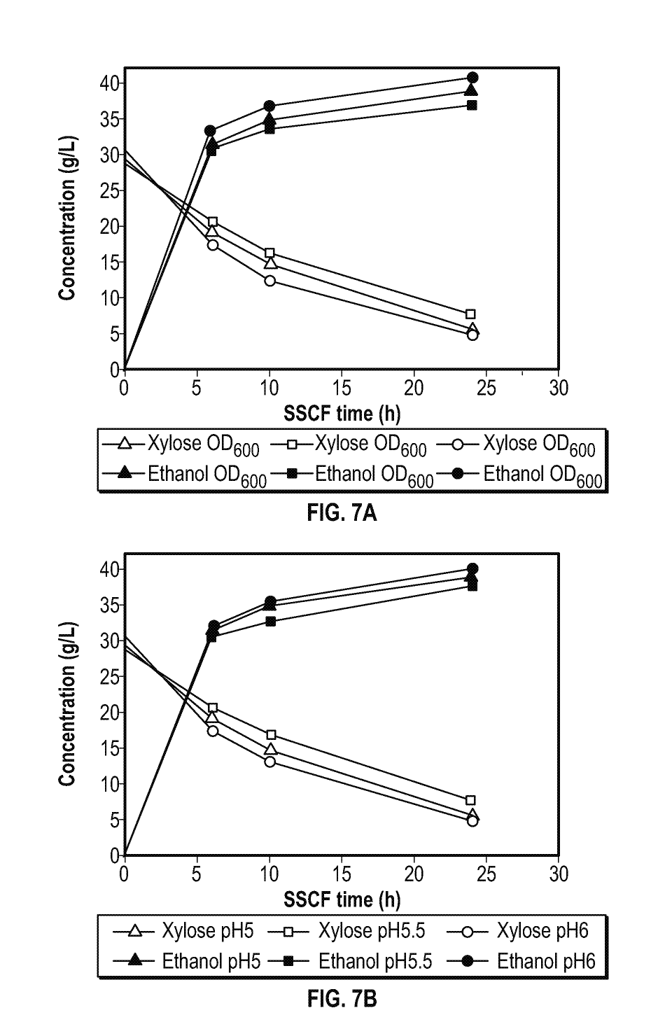

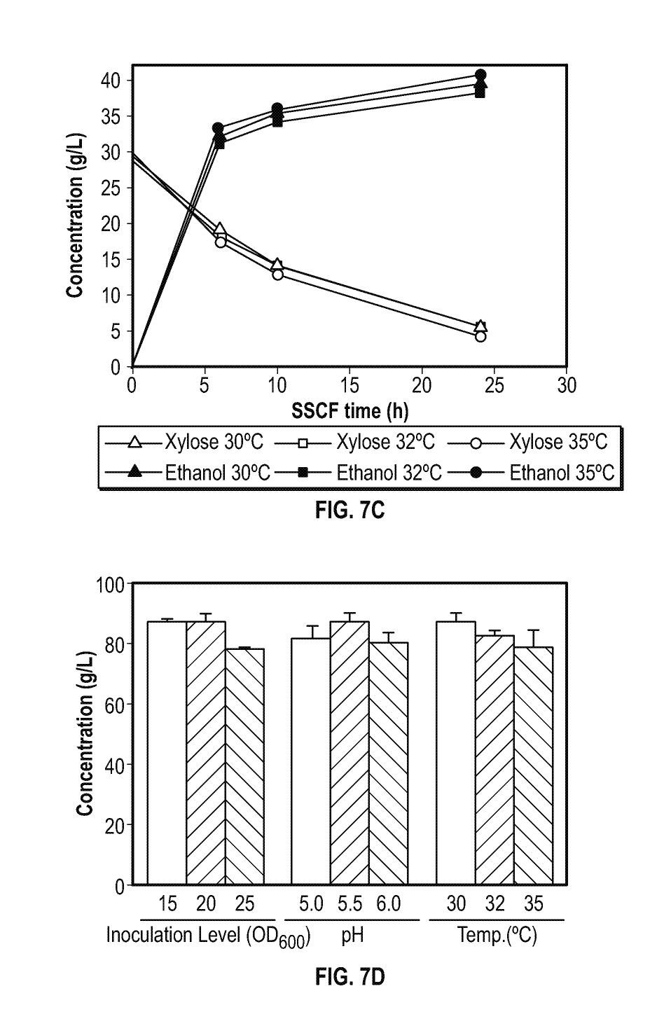

The initial OD, pH and temperature were optimized for the process of 24 h enzymatic hydrolysis followed by 24 h SSCF at 7% glucan loading. From FIG. 7A it is clear that by increasing the OD₆₀₀, more xylose was consumed and more ethanol was produced. However, the highest OD₆₀₀ tested (OD₆₀₀=25) showed lower cell viability (FIG. 7D). The fact that this yeast strain cannot grow well on xylose anaerobically (likely due to redox imbalance) and that the glucose release rate during SSCF might not support a high enough cell growth rate to balance the cell death rate can explain this result.

The best xylose fermentation was achieved at a pH of 6.0 (FIG. 7B). Nevertheless, the cell viability was not as good at this pH as it was at pH 5.5 (FIG. 7D) and it was also far removed from the optimum enzymatic hydrolysis pH 4.8, which would also affect the overall sugar conversion. A temperature of 32° C. produced more than 40 g/L ethanol with reasonable cell viability. The higher ethanol yield might be due to higher sugar conversions at higher temperature. However, the ethanol metabolic yield is lower at 35° C. as is the cell viability (FIG. 7D), which might explain the lower ethanol yield at 35° C. (FIG. 7C). Therefore, an initial OD₆₀₀ of 20, pH 5.5 and a temperature of 32° C. were chosen in an effort to obtain optimum conditions for the integrated FSSCF process. The same conditions were used for the integrated SHF process.

### Optimization of Enzyme Loading for Cycle 2 of Integrated SHF Process

To ensure that 40 g/L ethanol was produced, a 7.5% glucan loading was applied for the integrated SHF process. In this testing, unhydrolyzed solids, together with bound or adsorbed enzymes, were recycled for subsequent cycles of hydrolysis. With this approach, enzyme loading was reduced for cycles 2 through 5.

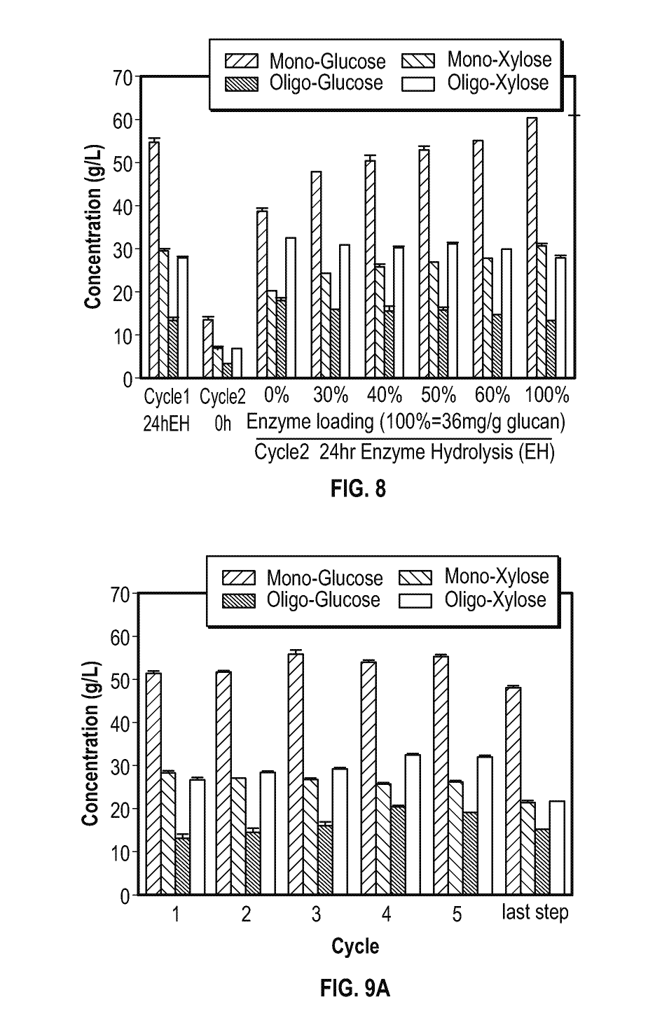

Experiments were then carried out to optimize the enzyme loading for cycle 2 of the integrated SHF process to obtain similar sugar concentrations as cycle 1 (FIG. 8). Without adding additional enzymes, 38.7 g/L glucose and 20.2 g/L xylose were obtained after 24 h enzymatic hydrolysis during cycle 2, which meant that the recycled enzymes from cycle 1 were functioning well. About 60% of the cycle 1's enzyme loading applied in cycle 2 yielded similar sugar concentrations as were obtained in cycle 1. Therefore, this enzyme loading (21.6 mg enzyme protein per glucan) was used for cycle 2, resulting in a 40% enzyme saving. Since the enzymes were being recycled and were likely accumulating, 50% of the cycle 1 enzyme loading (18 mg enzyme protein per glucan) was used for cycles 3 to 5.

### Integrated SHF Process Results

All the conversions were calculated based on total input glucose or total input xylose. During fermentation, there was still a certain amount of oligomeric glucose/xylose converted to monomeric glucose/xylose (Mono-Glucose/Mono-Xylose conversion). The Glucose-Ethanol/Xylose-Ethanol conversion was calculated based on total fermented glucose/xylose.

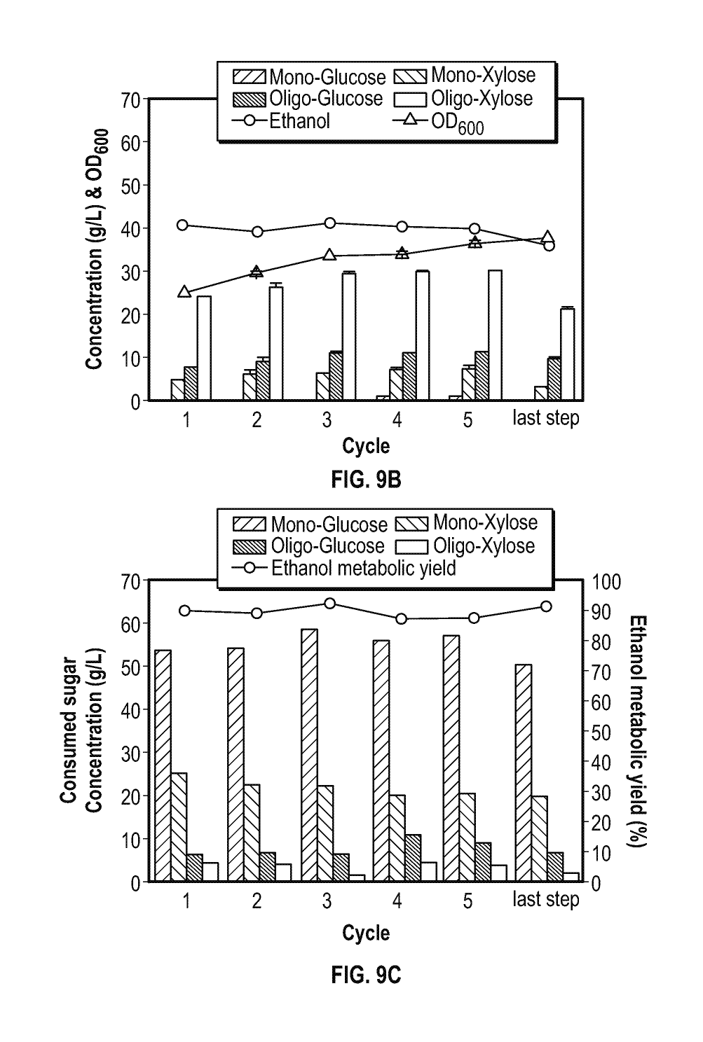

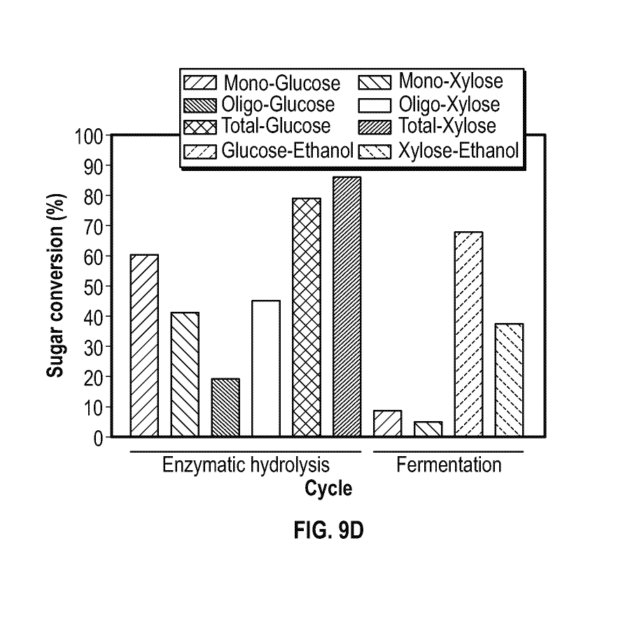

By applying the enzyme loadings discussed above and a glucan loading of 7.5%, consistently high sugar concentrations were produced for cycles 1 to 5 (FIG. 9A). The last step produced lower sugar concentrations compared to other cycles due to no addition of fresh enzymes or fresh biomass. It is likely that 24 h was too short a time for the hydrolysis of the final residual solids, and that these solids were more difficult to hydrolyze. Regardless, the removal of degradation products with liquid in previous cycles and lower sugar concentrations present at the beginning of the last step helped achieve reasonable sugar conversions from the non-easily digestible portion of the biomass which take the most cycles to hydrolyze.

Ethanol produced in each cycle was maintained at approximately 40 g/L and the OD increased from cycle to cycle (FIG. 9B), which guaranteed “fast” fermentation of xylose (FIG. 9C), i.e., in less than 24 h. The ethanol metabolic yield was maintained at about 90% (FIG. 9C). Although this is an SHF process, the enzymes (mostly β-Glucosidase and β-Xylosidase) present in the hydrolysate continued to function. As a result, a portion of the oligomeric sugars was converted to monomeric sugars during the fermentation process and then fermented to ethanol (FIG. 9C).

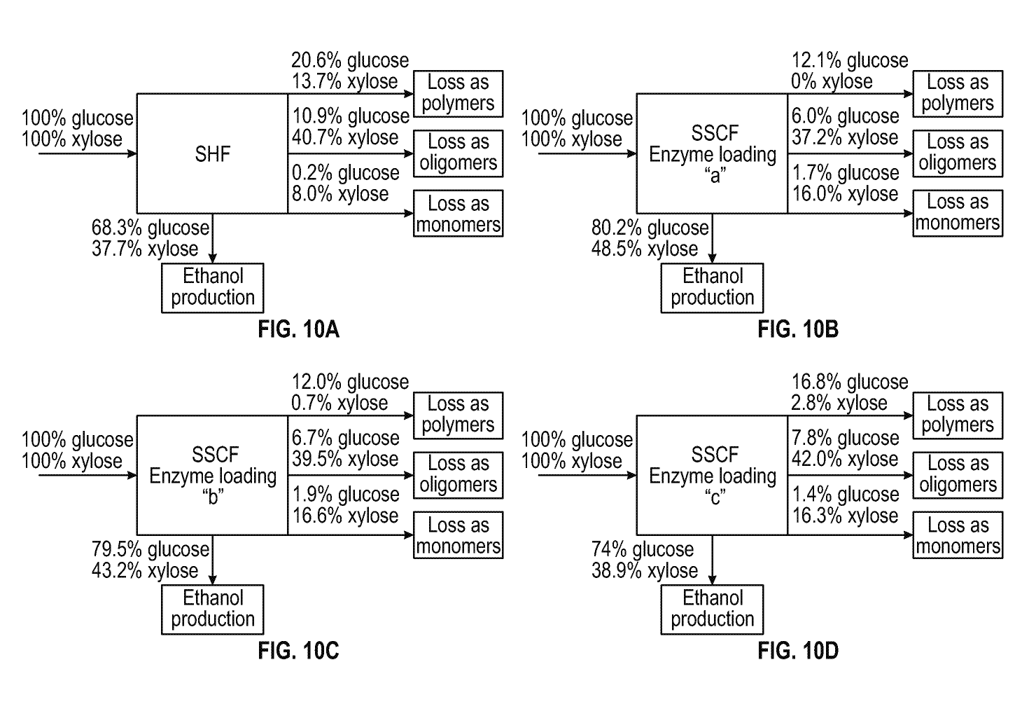

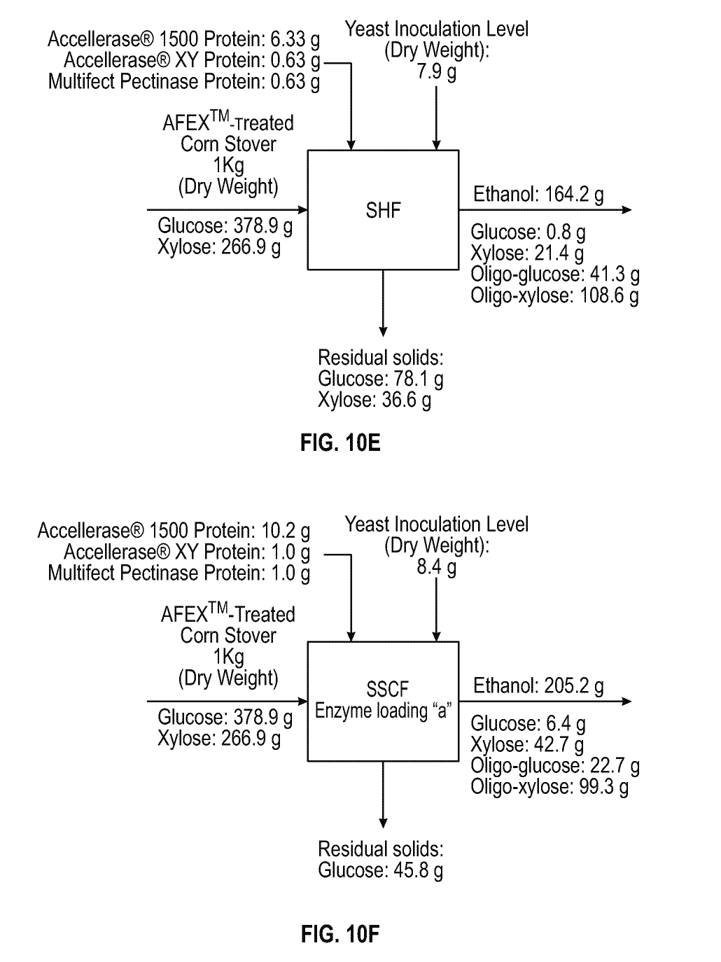

The overall glucan conversion and xylan conversion were 79.4% and 86.3%, respectively for this process (FIG. 9D). However, the glucose to ethanol and xylose to ethanol conversions were only 68.3% and 37.7%, respectively. The major sugar loss was due to the oligomeric sugars (see, for example, FIG. 10A), especially xylose oligomers (FIG. 9C), which cannot be consumed by yeast strains. It is expected that improved enzymes or engineered yeasts able to consume oligomers would help resolve this problem.

FIGS. 10A-10D show representative mass balances of processes utilizing 100% glucose and 100% xylose, including an integrated SHF process (FIG. 10A), an integrated FSSCF process with enzyme loading “a” (FIG. 10B), an integrated FSSCF process with enzyme loading “b” (FIG. 10C), and an integrated FSSCF process with enzyme loading “c” (FIG. 10D). As can be seen, the more enzymes loaded for the FSSCF process the more sugar was produced and converted to ethanol. A large amount of xylose in the form of sugar oligomers was not converted to ethanol due the particular microbial strain used in this example, which cannot consume oligomeric sugars. It is expected that use of a microbial strain capable of consuming oligomeric sugars can substantially further increase the ethanol yield.

FIGS. 10E and 10F show mass balances of processes utilizing a commercial protein blend and yeast inoculation with AFEX™-Treated Corn Stover in an integrated SHF process (FIG. 10E) and an integrated FSSCF process with enzyme loading “a” (FIG. 10F). As can be seen, the integrated SHF process has more potential to reduce the enzyme loading while the integrated FSSCF provided a higher ethanol yield.

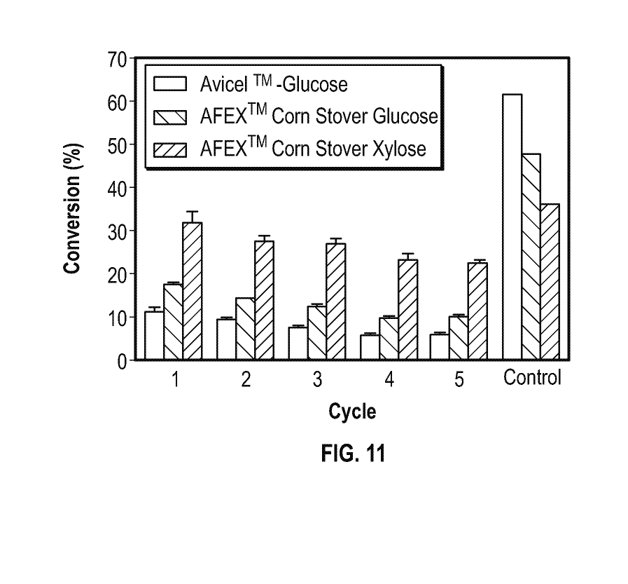

The assay was performed on Avicel™ and AFEX™-CS. Monomeric glucose/xylose conversion is shown in FIG. 11. The protein concentrations in each cycle hydrolysate were 3.2, 2.7, 2.6, 3.2 and 2.8 mg/ml. The control was the one assuming all the enzyme proteins were in the removed hydrolysate after cycle 1. The protein concentration for the control was 5.2 mg/ml. The enzyme activity assay was conducted in micro-plates with working volume 1.5 ml/well, substrate loading 1 mg/well, protein loading 30 ug/well, reaction time 12 h.

Large amounts of xylose oligomers exist in AFEX™ hydrolysates at high solid loadings, e.g., greater than 15 g/L. In addition, the enzyme loading ratio (cellulases:xylanases:pectinases) was the same for each cycle of the process. However, by analyzing the enzymes in the liquid hydrolysate for each cycle, it was determined that more β-glucosidase and xylanase were removed or denatured as compared to cellulases (FIG. 11). This result means that more xylanase and β-glucosidase should have been supplemented during late cycles and therefore the enzyme loading can likely be further reduced. Optimization of the enzyme cocktail for each cycle of this process may be performed to determine the overall potential for enzyme saving using the integrated process.

### Integrated FSSCF Process Results

Since the integrated FSSCF process recycles solids and enzymes to the next cycle of the SSCF tank, the enzyme loading can be reduced during later cycles. However, as the strain cannot tolerate high temperatures and the cells were recycled along with the enzymes, the recycled enzymes work at the fermentation temperature (32° C.). Therefore, the enzyme reduction achieved in the integrated FSSCF process is less than it is for the integrated SHF process.

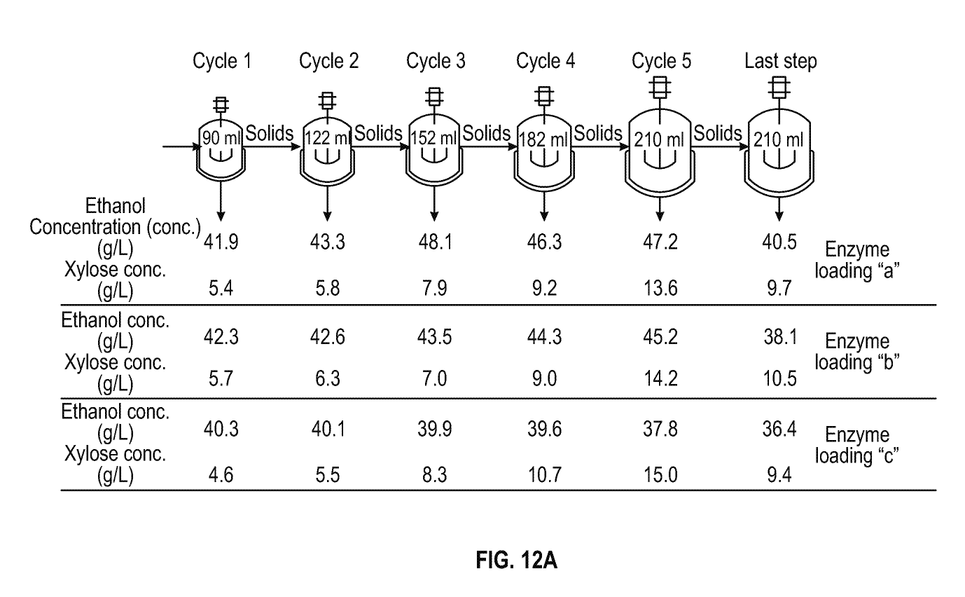

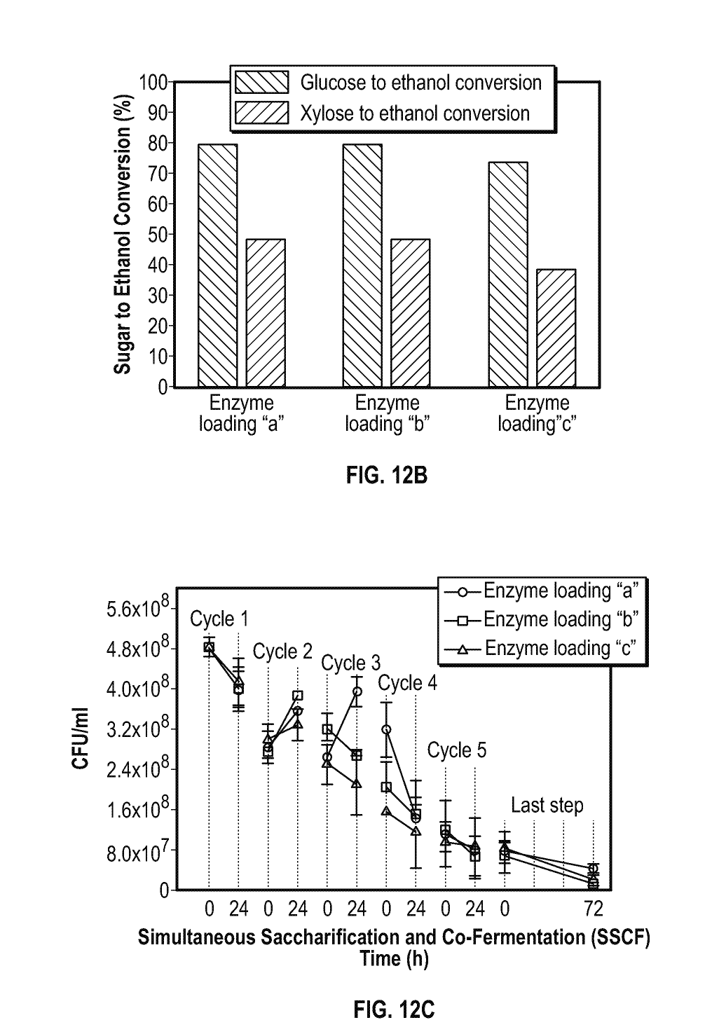

FIGS. 12A-12C show the effect of enzyme loading profile on ethanol production (FIG. 12A), sugar to ethanol conversions (FIG. 12B), and viable cell density FIG. 12C) during an integrated FSSCF process. Three enzyme loading profiles were tested as shown in FIGS. 12A-12C. Enzyme loading “a”=36 mg/g glucan for each cycle; Enzyme loading “b”=36.0 (100%), 32.4 (90%), 30.6 (85%), 28.8 (80%) and 27.0 (75%) mg/g glucan for cycles 1 through 5, respectively, with an average enzyme loading of 30.9 mg/g glucan; Enzyme loading “c”=30.0 (100%), 27.0 (90%), 25.5 (85%), 24.0 (80%) and 22.5 (70%) mg/g glucan for cycles 1 through 5, respectively, with an average enzyme loading of 25.8 mg/g glucan. The ratio of enzymes cocktails applied (cellulases:xylanase:pectinase) was fixed at approximately 10:1:1.

The ethanol concentration for each cycle largely met the criterion of 40 g/L for all three cases (FIG. 12A). Glucose to ethanol conversions were about 75% to about 80% (FIG. 12C). Xylose to ethanol conversions were about 40% to about 50%. Most of the xylose was in oligomeric form and was not available for fermentation.

Decreasing viable cell densities were observed from cycle to cycle (FIG. 12C), which caused a buildup of xylose (xylose loss as monomers) during later cycles (FIGS. 12C and 10A-D). The solid residue concentrations in cycles 1 to 6 of the integrated FSSCF process were about 88, about 125, about 153, about 170, and about 185 g/L, respectively.

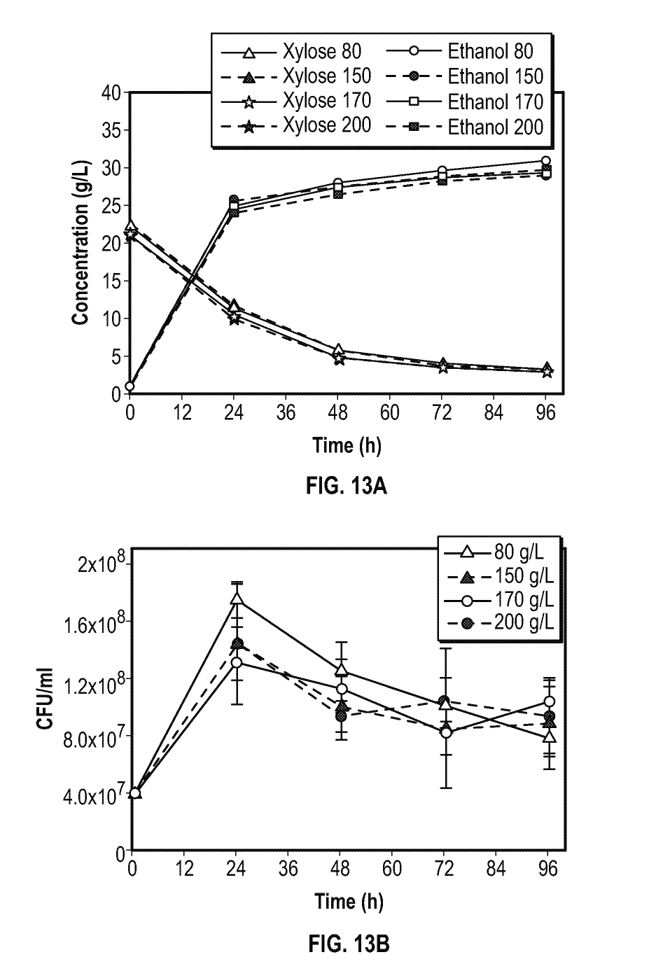

FIGS. 13A and 13B show the effect of enzymatic hydrolysis residual solids concentration over time on fermentation in the digested solids (i.e., hydrolysate). Solids concentration investigated included about 80, about 150, about 170, and about 200 g/L. Fermentations were performed in 250 ml shake flasks with about 100 ml working volume at 180 rpm, 32° C., pH 5.5 and initial OD₆₀₀ of 2.0. The initial glucose concentration was 43.8±1.4 g/L and consumed completely in 24 h for all of the cases. The solids concentrations in cycle 1-6 of the integrated FSSCF process were about 88, about 125, about 153, about 170, about 185, and about 185 g/L, respectively. It is possible that high solids concentration can lead to (FIGS. 13A-13B) higher biofuel production.

### Comparisons of Process Ethanol Productivity and Ethanol Yield of Different Process

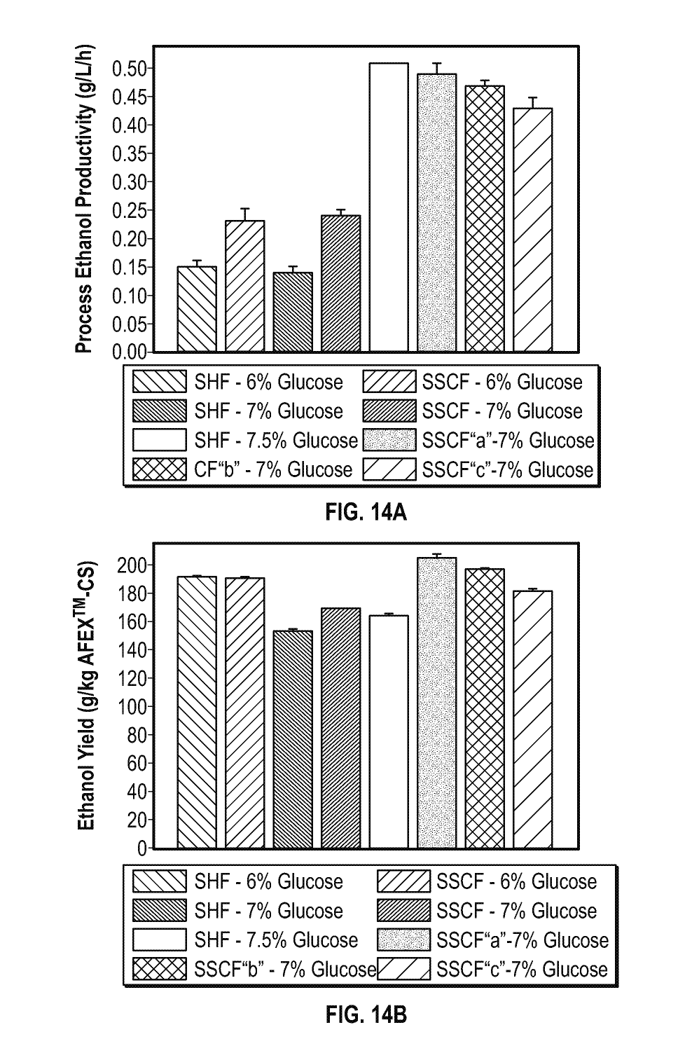

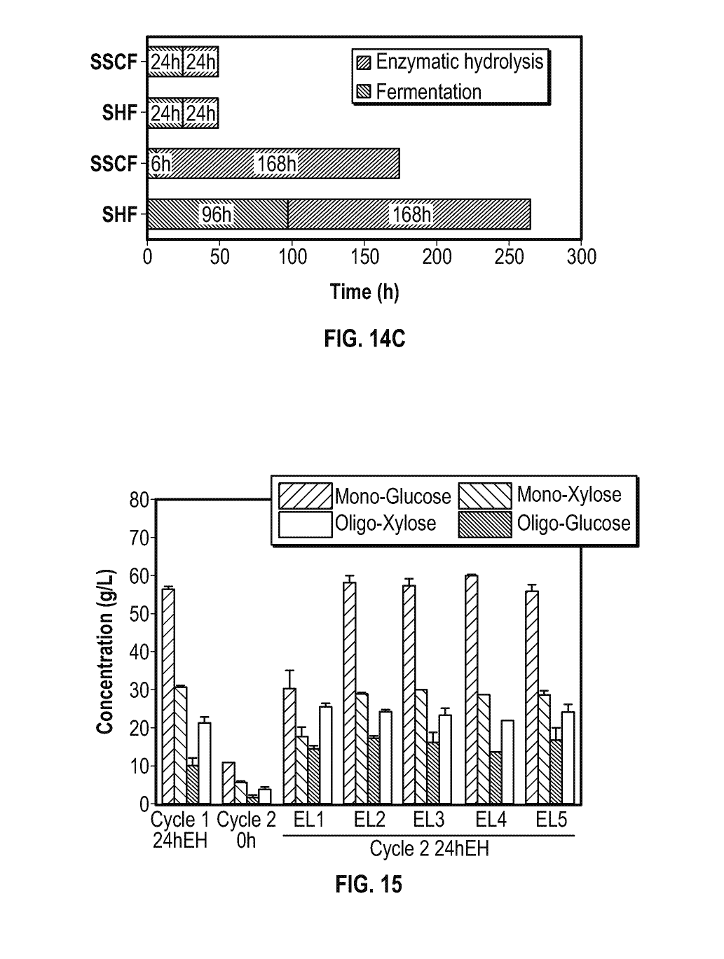

The integrated SHF and FSSCF processes showed much higher process ethanol productivities compared to conventional SHF or SSCF processes (FIG. 14A) probably due to the time saved (FIG. 14C) by fast hydrolysis and fast fermentation. In addition, the series of stirred tanks used for enzymatic hydrolysis behaved like a plug flow reactor. The unhydrolyzed solids, which were typically non-easily digestible, flowed from one EH reactor to another. Thus the conversion of those solids was dependent on the position in the reactor train rather than time. Plug flow reactors are well known for possessing higher average reaction rates for positive order reactions than do stirred tanks. Therefore, using a series of stirred tanks was likely another factor giving higher ethanol productivity.

SHF results were based 96 h enzymatic hydrolysis and 168 h fermentation (c). SSCF results were based on 6 h pre-hydrolysis and 168 h fermentation. Enzyme loadings for these processes were at approximately levels of 36, 36, 36, 36, 22.3, 36, 30.9, and 25.8 mg/g glucan, respectively. GL: glucan loading. FIGS. 14A-C shows comparison of ethanol productivity from a SHF process described herein and a SSCF process. The integrated processes showed much higher ethanol productivity (FIG. 14A) compared to conventional processes with lower enzyme loadings and similar ethanol yields (FIG. 14B). The higher productivity is most likely due to the much shorter time needed for the integrated processes (FIG. 14C).

For the ethanol yield, 7% glucan loading was less effective than a 6% glucan loading using conventional processes (FIG. 14B). This might due to the inhibitory effect of degradation products on both enzymatic hydrolysis and fermentation (See FIGS. 6A and 6B). The integrated SHF process at 7.5% glucan loading showed similar ethanol yields compared to conventional processes at similar glucan loadings but with much lower enzyme loading (22.3 vs. 36 mg protein/g glucan). The integrated FSSCF process showed higher or similar ethanol yields (depending on enzyme loading) at 7% glucan loading compared to conventional fermentation processes at 7% or 6% glucan loading.

### Example 2

### Extractive AFEX™ Pretreated Corn Stover (E-AFEX™-CS)

E-AFEX™-CS with glucan and xylan contents of 43.3% and 26.7%, respectively, acid insoluble lignin content of 9% and acetyl content of 1%, from GLBRC (Great Lakes Bioenergy Research Center) was used. E-AFEX™ pretreatment conditions were as follows: Ammonia:biomass ratio of 6:1, moisture content 10%, Temp: 120° C., Time: 1 h, Pressure: 88.46 atm (300 psi).

### Microorganism

The xylose-fermenting strain Saccharomyces cerevisiae 424A(LNH-ST) was provided by Purdue University in Indiana. LNH-ST is a genetically modified strain with multiple copies of xylose reductase (XR) and xylitol dehydrogenase (XD) genes from Scheffersomyces (Pichia) stipitis and an endogenous xylulokinase gene (XK) incorporated in the chromosome. The seed culture of this strain was prepared in a 250 ml Erlenmeyer flask with 100 ml YPDX medium (10 g/L yeast extract, 20 g/L peptone, 75 g/L glucose and 20 g/L xylose).

### Enzymatic Hydrolysis

Enzymatic hydrolysis (EH) was conducted in a 250 ml or 500 ml baffled flask. Hydrolysis conditions were 50° C., pH 4.8 and 250 rpm.

### Fermentation

Fermentation of hydrolysate was performed at 32° C., pH 5.5 and 150 rpm. Glucose, xylose and ethanol concentrations were analyzed by HPLC using a Biorad Aminex HPX-87H column. Column temperature was maintained at 50° C. Mobile phase (5 mM H₂SO₄) flow rate was 0.6 mL/min.

### Results

According to the study on regular AFEX™-pretreated CS, more xylanases than cellulases were lost with removing liquid hydrolysate. As such, the combination of enzymes for Cycle 2 is optimized. See Table 1.

TABLE 1
Experimental Design for Optimization of Total
Enzyme Loading (EL) for Cycle 2.

| Cycle-Total | Total EL mg protein/ | | | |
|---|---|---|---|---|
| EL No. | g glucan | Ctec2 | Htec2 | Pectinase |
| 1 | 12 | 8 | 2 | 2 |
| 2-EL1 | 0 | 0 | 0 | 0 |
| 2-EL2 | 7.5 | 5 | 1.25 | 1.25 |
| 2-EL3 | 7.5 | 3.75 | 1.88 | 1.88 |
| 2-EL4 | 7.5 | 6 | 0.9 | 0.6 |
| 2-EL5 | 6 | 4 | 1 | 1 |

Enzyme loadings for the Cycle 1 through Cycle 5 and the last step during fast SHF process are shown in Table 2.

TABLE 2
Total EL for Cycle 1-Cycle 5 and Last step During
Integrated SHF Process

| | Total EL (mg | | | |
|---|---|---|---|---|
| | protein/g glucan) | Ctec2 | Htec2 | Pectinase |
| Cycle 1 | 12 | 8 | 2 | 2 |
| Cycle 2 | 7.5 | 5 | 1.25 | 1.25 |
| Cycle 3 | 6 | 4 | 1 | 1 |
| Cycle 4 | 6 | 4 | 1 | 1 |
| Cycle 5 | 6 | 4 | 1 | 1 |
| Last Step | 2.5 | 2 | 0.25 | 0.25 |
| Average | 8 | 5.4 | 1.3 | 3 |

These results show that an integrated SHF process generated approximately 181 g ethanol from about 1 kg E-AFEX™ pretreated corn stover with an average enzyme loading of 8 mg/g glucan and overall productivity of about 0.46 g/L/h. Additionally, the ethanol metabolic yield during fermentation (about 0.43 g ethanol/g consumed sugar) was lower compared to previous fermentations, which were about 0.47 g ethanol/g consumed sugar. Additional testing can confirm these results. In one embodiment, the last step of enzymatic hydrolysis can be further optimized.

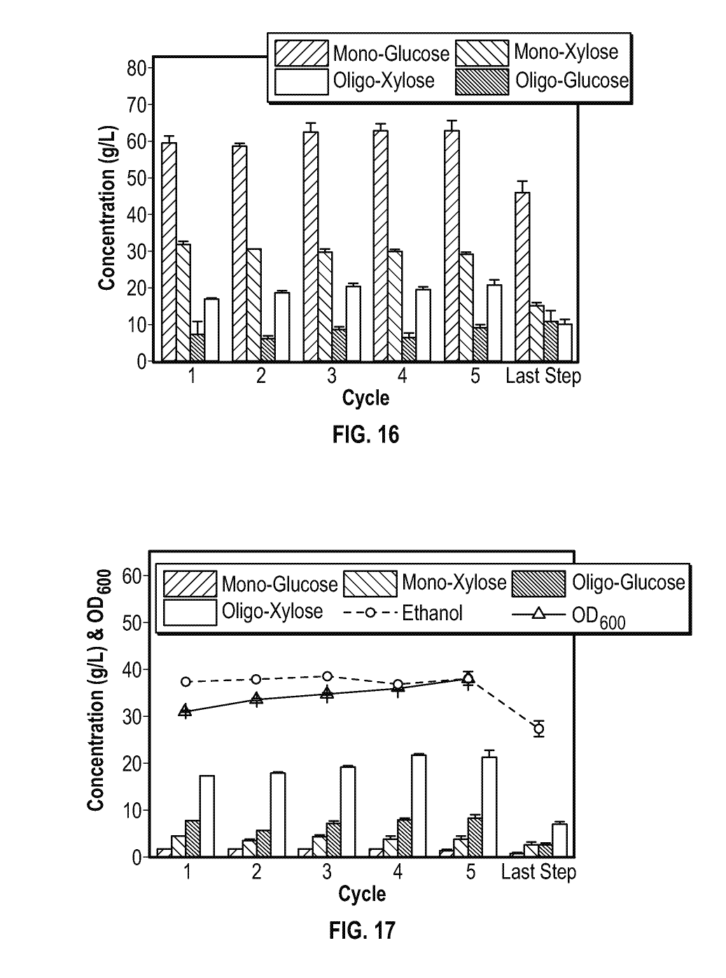

FIG. 15 shows the optimization of enzyme loading for Cycle 2. As can be seen, different combinations of enzymes (EL1, EL2 and EL3) show substantially the same sugar yield. See also FIGS. 16 and 17, which show enzymatic hydrolysis and fermentation, respectively, for an integrated SHF process. Consistent sugar concentrations were produced from cycle 1˜5 even with enzyme loading reduced by half in the cycles of 3 through 5 (FIG. 16), which provided consistent ethanol production from cycle to cycle (FIG. 17).

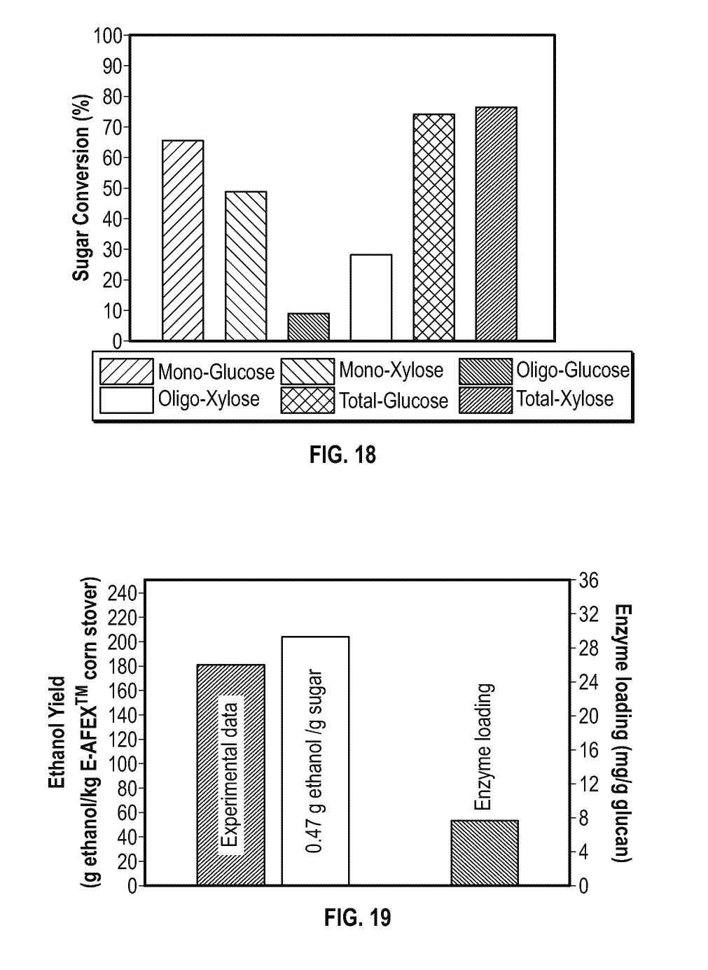

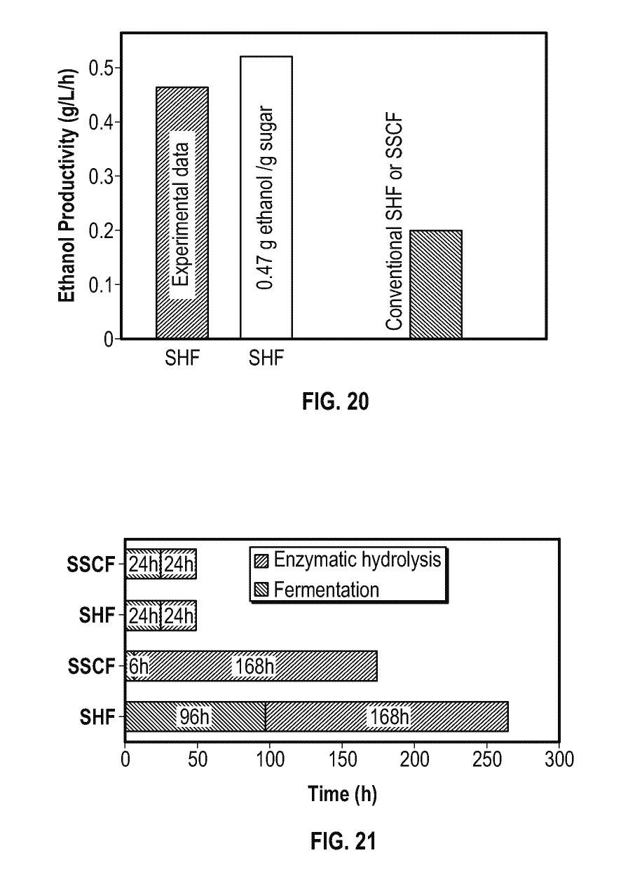

FIGS. 18-21 are graphs showing sugar conversion (FIG. 18), ethanol yield (FIG. 19), ethanol productivity (FIG. 20) and time scale (FIG. 21) of an integrated SHF process on E-AFEX™ pretreated corn stover. FIGS. 18-21 were adapted from information in Applications '569 and '408. For purposes of FIGS. 18-21, it was assumed that the ethanol metabolic yield was approximately 0.47 g/g of consumed sugars, which was the typical value obtained. The lower ethanol metabolic yield shown in the experimental data was likely due to the properties of the pretreated biomass. On the E-AFEX™ pretreated corn stover, the integrated processes also increased the ethanol productivity by around 2˜3 fold with enzyme loading reduced by ⅓.

The enzyme recycling depends on the enzyme adsorption to the residual solids. It is possible that high sugar concentrations in the integrated SHF process inhibited enzyme adsorption, thus affecting enzyme recycling. Although the integrated FSSCF process removed most of the sugars by fermentation, this process produced ethanol, which might have an effect on enzyme adsorption.

The integrated FSSCF process achieved higher ethanol yield compared to the integrated SHF process probably because of the additional 24 h enzymatic hydrolysis during fermentation even though the hydrolysis was not performed at its optimal conditions.

High productivity achieved by integrated processes can potentially reduce the reactor sizes and, therefore, reduce both the capital costs and operating costs.

### Example 3

### Prophetic

Enzyme mixtures for AFEX™-CS described in D. Gao, et. al., Bioresource Technology, 2010, 101, 2770-2781, which is incorporated herein by reference in its entirety, generate concentrations of monomeric sugars (including xylose) which are higher than the concentrations attainable with commercial enzymes, such as the one used in Example 1. As such, additional testing will be performed using higher-capacity equipment together with a sufficient amount of purified enzymes.

### Example 4

### Prophetic

Integrated FSSCF testing will be performed with an microbial strain having increased thermo-tolerance as compared to the microbial strain described herein (e.g., Kluyveromyces marxianus and Thermoanaerobacterium saccharolyticum).

The various embodiments provide for a method comprising performing a first hydrolyzing cycle on lignocellulosic biomass (e.g., corn stover (CS), switchgrass rice straw and combinations thereof) containing an easily digestible portion (e.g., hemicellulose) and a non-easily digestible portion, to produce a slurry containing digested solids and unhydrolyzed solids, respectively; and producing a solids residue and a liquid coproduct from the slurry. In one embodiment, the method further comprises recycling at least a portion of the solids residue through a second hydrolyzing cycle, wherein an amount of the undigested solids in the solids residue is converted to digested solids. Various embodiments can further comprise performing additional hydrolyzing cycles to produce increasing amounts of digested solids.

In one embodiment, the easily digestible portion comprises hemicellulose and the non-easily digestible portion comprises lignin and/or cellulose.

In one embodiment, substantially all the non-easily digestible portion produced after the first cycle is substantially hydrolyzed after the second cycle and/or the digested solids are comprised predominantly of hydrolyzed solids, such as, but not limited to, hydrolyzed sugars selected from monomeric sugars, oligomeric sugars, xylo-oligomeric sugars and combinations thereof.

In one embodiment, the pretreatment is an ammonia pretreatment process, such as an ammonia fiber expansion process.

In various embodiments, enzyme loading can be reduced by at least 1% up to 39%, including any range there between. In various embodiments, fermentation is completed in less than 24 hours.

Any suitable yeast inoculation level can be used. In one embodiment, the yeast inoculation level is at least 10 times up to about 40 times higher as compared to a conventional non-integrated hydrolysis process with no recycle steps. In one embodiment, the yeast inoculation level is at least 2 OD. In one embodiment, the yeast inoculation level is greater than 2 OD.

In one embodiment, a system is provided comprising a first vessel for performing a first hydrolyzing cycle on lignocellulosic biomass containing an easily digestible portion and a non-easily digestible portion to produce a slurry containing digested solids and unhydrolyzed solids, respectively; and a second vessel for producing solids residue and a liquid coproduct from the slurry; and a bioproduct production facility connected to the integrated hydrolysis system and configured to perform either separate hydrolysis and fermentation (SHF) or simultaneous saccharification and co-fermentation (SSCF).

In one embodiment, the system is an integrated hydrolysis system which further comprises a recycling system configured to perform recycling of the solids residue through a second hydrolyzing cycle, wherein an amount of the undigested solids in the solids residue can be converted to digested solids.

In various embodiments, an additional hydrolyzing cycle is performed in the first vessel to produce increasing amounts of digested solids.

The bioproduct production facility can be, in various embodiments, a biofuel production facility or a biochemical production facility. The biofuel production facility can be an alcohol production facility, such as an ethanol or butanol production facility.

In one embodiment, an apparatus is provided comprising integrated hydrolysis equipment configured to perform a first hydrolyzing cycle on lignocellulosic biomass containing an easily digestible portion and a non-easily digestible portion to produce a slurry containing digested solids and unhydrolyzed solids, respectively, wherein the equipment is further configured to perform a production step, in which solids residue and a liquid coproduct are produced from the slurry, and a recycling step, in which the solids are recycled through a second hydrolyzing cycle, wherein an amount of the undigested solids in the solids residue is converted to digested solids.

The integrated hydrolysis equipment can include, in various embodiments, one or more vessels, at least one fermenter and at least one saccharification tank. In one embodiment, the integrated hydrolysis equipment includes an equal number of fermenters and saccharification tanks.

In one embodiment, increased sugar production rates result in increased bioproduct production rates.

Using the NREL 2011 model as a guide (D. Humbird, et al.), it can be estimated that if conventional SHF process performance on AFEX™-CS utilize “x” number of saccharification tanks, each having a capacity of about 946,353 Liters (L) (250,000 gallons (gal)) and 3·x the number of fermenters, each having a capacity of about 3,785,412 L (1,000,000 gal), to process a given amount of corn stover per day. For example, about 12 such saccharification tanks and about 36 such fermenters can be used to process about 2,205 dry U.S. ton/day (2,000 metric tonne/day) of corn stover per day.

In contrast, use of one embodiment of an integrated SHF described herein can reduce the number of such fermenters to less than 3 times the number of saccharification tanks, down to less than 2 times the number of saccharification tanks, down to about the same number of saccharification tanks as fermenters. In one embodiment, the same number of saccharification tanks and fermenter tan are used. In one embodiment, use of the same number of saccharification tanks and fermenters results in a reduction in total reaction volume being treated by up to about 62%.

In one embodiment, the cost of centrifugation and filtrations for the integrated processes are comparable to costs for conventional processes. Therefore, in one embodiment, approximately 62% of the capital cost in saccharification tanks, fermenters and their accessories can be saved.

The cost of enzymes alone account for approximately 16% of the total ethanol production cost (NREL 2011 model). In one embodiment, the integrated SHF process uses at least about 10% up to about 20% up to about 30% up to about 40% (including any range there between) less enzymes as compared with conventional processes. In one embodiment, the reduction is between about 35 and about 45% such as between about 37 and about 39%. In one embodiment, at least a 38% reduction in enzyme use is obtained as compared to conventional process, thus reducing enzyme cost by comparable amounts.

The novel integrated biological processes described herein save time by fermenting xylose more quickly than conventional processes and, in one embodiment, process the easily digestible biomass first to fully utilize the high enzymatic hydrolysis rate period, and hydrolyze the more non-easily digestible portion of biomass with less inhibition by degradation products and sugars, thereby enhancing the process ethanol productivity. In various embodiments, biocatalyst resources are better utilized by recycling yeast cells and enzymes, thereby reducing the overall processing cost.

In one embodiment, enzyme loading can be reduced by more than 10%, up to about 20% up to about 25% up to about 30% up to about 35% or higher, such as at least 33%. In one embodiment, fermentation can be substantially completed within 24 h or less. In one embodiment, ethanol productivity can be increased by at least about two- to three-fold.

Although specific embodiments have been illustrated and described herein, it will be appreciated by those of ordinary skill in the art that any procedure that is calculated to achieve the same purpose may be substituted for the specific embodiments shown. This application is intended to cover any adaptations or variations of the present subject matter. For example, although the various embodiments have been described with respect to biofuels, such as ethanol, it is understood that other bioproduct production processes, such as biochemical production processes, may benefit. Therefore, it is manifestly intended that embodiments of this subject matter be limited only by the claims and the equivalents thereof.

What is claimed is:

1. A method comprising:
performing a first hydrolyzing cycle on lignocellulosic biomass containing an easily digestible portion and a non-easily digestible portion, to produce a slurry containing digested solids and unhydrolyzed solids, respectively; and
producing a solids residue and a liquid coproduct from the slurry.

2. The method of claim 1 further comprising recycling at least a portion of the solids residue through a second hydrolyzing cycle, wherein an amount of the undigested solids in the solids residue is converted to digested solids.

3. The method of claim 1 further comprising performing additional hydrolyzing cycles to produce increasing amounts of digested solids.

4. The method of claim 3 wherein substantially all the new easily digestible portion produced after the first cycle is substantially hydrolyzed after the second cycle and/or the digested solids are comprised predominantly of hydrolyzed solids.

5. The method of claim 4 wherein the hydrolyzed solids are hydrolyzed sugars selected from monomeric sugars, oligomeric sugars, xylo-oligomeric sugars and combinations thereof

6. The method of claim 1 wherein the easily digestible portion comprises hemicellulose, and the non-easily digestible portion comprises lignin and/or cellulose.

7. The method of claim 6 wherein the lignocellulosic biomass is selected from corn stover (CS), switchgrass, rice straw and combinations thereof.

8. The method of claim 1 wherein the pretreatment is an ammonia pretreatment process.

9. The method of claim 8 wherein the ammonia pretreatment process is ammonia fiber expansion process.

10. The method of claim 1 wherein enzyme loading is reduced by at least 1% up to 39%, including any range there between.

11. The method of claim 1 wherein fermentation is completed in less than 24 hours.

12. The method of claim 11 wherein a yeast inoculation level is at least 10 times up to about 40 times higher as compared to a conventional non-integrated hydrolysis process with no recycle steps.

13. The method of claim 12 wherein the yeast inoculation level (OD₆₀₀) is greater than 2.

14. A system comprising:
a first vessel for performing a first hydrolyzing cycle on lignocellulosic biomass containing an easily digestible portion and a non-easily digestible portion to produce a slurry containing digested solids and unhydrolyzed solids, respectively; and
a second vessel for producing solids residue and a liquid coproduct from the slurry; and
a bioproduct production facility connected to the integrated hydrolysis system and configured to perform either separate hydrolysis and fermentation (SHF) or simultaneous saccharification and co-fermentation (SSCF).

15. The system of claim 14 comprising an integrated hydrolysis system which further comprises a recycling system configured to perform recycling of the solids residue through a second hydrolyzing cycle, wherein an amount of the undigested solids in the solids residue can be converted to digested solids.

16. The system of claim 14 wherein the bioproduct production facility is a biofuel or biochemical production facility.

17. The system of claim 16 wherein the biofuel production facility is an alcohol production facility.

18. The system of claim 17 wherein the alcohol production facility is a butanol or ethanol production facility.

19. The system of claim 14 wherein an additional hydrolyzing cycle is performed in the first vessel to produce increasing amounts of digested solids.

20. An apparatus comprising:
integrated hydrolysis equipment configured to perform a first hydrolyzing cycle on lignocellulosic biomass containing an easily digestible portion and a non-easily digestible portion to produce a slurry containing digested solids and unhydrolyzed solids, respectively, wherein the equipment is further configured to perform a production step, in which solids residue and a liquid coproduct are produced from the slurry, and a recycling step, in which the solids are recycled through a second hydrolyzing cycle, wherein an amount of the undigested solids in the solids residue is converted to digested solids.

21. The apparatus of claim 20 wherein the integrated hydrolysis equipment includes one or more vessels, at least one fermenter and at least one saccharification tank.

22. The apparatus of claim 20 wherein the integrated hydrolysis equipment includes an equal number of fermenters and saccharification tanks.
